(19) United States
(12) Patent Application Publication
(10) Pub. No.: US 2014/0234269 A1
(43) Pub. Date: Aug. 21, 2014

(54) Endometrial Derived Stem Cells and Their Methods of Use

(71) Applicant: Hugh S. Taylor, Easton, CT (US)

(72) Inventor: Hugh S. Taylor, Easton, CT (US)

(73) Assignee: YALE UNIVERSITY, New Haven, CT (US)

(21) Appl. No.: 14/233,941

(22) PCT Filed: Jul. 20, 2012
(86) PCT No.: PCT/US12/47611

§ 371 Date: May 5, 2014

Related U.S. Application Data
(60) Provisional application No. 61/510,812, filed on Jul. 22, 2011.

(51) Int. Cl. C12N 5/071; A61K 35/39

(52) U.S. Cl. CPC C12N 5/0676; A61K 35/39; C12N 2506/13; C12N 2501/16; C12N 2501/119; C12N 2501/335; C12N 2501/11; C12N 2501/727; C12N 2501/385; C12N 2533/90; C12N 2500/34; C12N 2500/38
USPC 424/93.7; 435/377; 435/366

(57) ABSTRACT

The present invention provides a method of making an insulin-producing cell derived from an endometrial stromal stem cell (ESSC). The invention includes the progeny of ESSC, including any cell type generated during the differentiation of ESSC towards cells that produce insulin and exhibit cell markers characteristic of insulin producing cells. The cells of the invention can be used to treat various diseases such as diabetes type I, diabetes type II and gestational diabetes.

### CROSS-REFERENCE TO RELATED APPLICATIONS

This application claims priority to U.S. Patent Application Ser. No. 61/510,812, filed Jul. 22, 2011, the contents of which are incorporated by reference herein in its entirety.

### BACKGROUND OF THE INVENTION

Diabetes is a global epidemic that affects the lives of 171 million people world-wide (2.8%) (Rathmann et al., 2004, Diabetes Care 27:2568-2569). The disease prevalence is related to trends in population growth, aging, urbanization, obesity and physical inactivity. The main causes are loss of insulin production from pancreatic β-cells in the islets of Langerhans (type I) or resistance to insulin (type II).

Results from multiple studies have suggested that islet-based transplantation has potential as a clinical approach in the treatment of type I diabetes mellitus (Lumelsky et al., 2001, Science 292:1389-1394; Ramiya et al., 2000, Nat. Med. 6:278-282; Robertson, 2004, New Eng. J. Med. 350:694-705; Huang et al., 2008, Endocr. Rev. 29:603-630; Xu et al., 2008, Cell 132:197-207). However, the development of such therapy is still under investigation (Ahlgren et al., 1997, Nature 385:257-260; Noguchi, 2010, Curr. Diabetes Rev. 6:184-190; Tan et al., 2008, Diabetes 57:2666-2671) and not widely used due to the severe shortage of transplantable donor islets as well as tissue rejection (Street et al., 2004, Int. J. Biochem. Cell Biol. 36:667-683). One promising method to overcome donor-host rejection is autologous stem cell transplantation. In autologous stem cell therapy, the derivation of insulin-producing cells is accomplished by the induction to differentiation of the pluripotent or multipotent cells obtained from the patient. Pluripotent cells are self-renewing with the capability to give rise to all cell types. Currently they are derived from adult cells by reprogramming, as in the case of induced-pluripotent stem cells (Takahashi et al., 2007, Cell 131:861-872). However, induced-pluripotent stem cells are genetically altered and can form teratomas, introducing clinical risks yet to be resolved. Adult multipotent stem cells, such as mesenchymal stem cells are self-renewing cells that give rise to specific cell lines and which originated in the embryonic mesenchyme. Isolated mesenchymal stem cells from numerous tissues, such as the bone marrow stroma (Oh et al., 2004, Lab. Invest. 84:607-617), the umbilical cord (Gao et al., 2008, Chin. Med. J. 121:811-818) or the amnion (Tamagawa et al., 2009, Hum. Cell 22:55-63), have shown the capacity to differentiate in vitro and in vivo into multiple cell lines and across all three germ layers. In comparison to induced-pluripotent stem cells, mesenchymal stem cells are considered relatively safer for therapeutic purposes and several are currently used in clinical trial for numerous indications. Nevertheless, the use of multipotent stem cells has barriers. Access to matched umbilical cord and amniotic stem cells is limited to those who stored this tissue at birth. Bone marrow biopsy is painful and requires general anesthesia. Therefore, there is still demand for a source of allogenic multipotent stem cells that are easily obtainable, practical, and safe.

The human endometrium is a highly dynamic regenerative tissue that undergoes a mean of 400 cycles throughout the woman's fertile lifespan. This tissue rapidly regenerates in response to estrogen even after menopause. Endometrial biopsy is a simple method to obtain an extensive supply of endometrial cells from a simple office procedure. In addition, approximately 600,000 hysterectomies are yearly performed in the United States, creating another potential source of endometrial cells (Schwab et al., 2008, Human Reproduction 23:934-943). Recently, it was shown that endometrial stem cells have the capacity to differentiate into several mesodermal and ectodermal cell lineages including condrocytes, adipocytes, myocytes, and osteocytes (Schwab et al., 2008, Hum. Reprod. 23:934-943; Gargett et al., 2009, Biol. Reprod. 80:1136-1145; Wolff et al., 2007, Reprod. Sci. 14:524-233). The ability to generate dopamine producing neurons from adult human endometrial stromal stem cells (ESSC) as well as successful transplant and function in an animal model of Parkinson's disease has previously been demonstrated (Wolff et al., 2011, J. Cell Mol. Med. 15:747-755). However, differentiating endometrial stem cells into pancreatic β-cells, which involves a shift between the two lineage fates, has yet to be achieved.

The pancreatic endocrine compartment mainly consists of islets of Langerhans, which are composed of four cell types that synthesize peptide hormones such as insulin (β-cells), glucagon (α-cells), somatostatin (δ-cells) and pancreatic polypeptide (γ-cells). These cells originate from endoderm and have been shown to rise from ductal epithelium through sequential differentiation during embryogenesis (Hellerström, 1984, Diabetologia 26:393-400; Seeberger et al., 2006, Lab. Invest. 86:141-153; Zaret et al., 2008, Science 322:1490-1494). Due to their accessibility and ability to regenerate rapidly in response to estrogen, ESSC are an excellent candidate for use in stem cell therapies.

There is a need in the art for cell therapies using ESSC and their progeny. The present invention addresses this unmet need in the art.

### SUMMARY OF THE INVENTION

The present invention provides a method of making an insulin-producing cell derived from an endometrial stromal stem cell (ESSC).

In one embodiment, the method comprising the steps of: a) contacting at least one ESSC with a first cell culture medium comprising 20-30 mmol/l glucose, 5-15% FBS and 10⁻⁵-10⁻⁷ mol/l retinoic acid, and incubating the at least one cell for about 12-36 hours; then b) contacting the at least one ESSC with a second cell culture medium comprising 20-30 mmol/l glucose and 5-15% FBS and incubating the at least one ESSC for about 1-4 days; then c) contacting the at least one ESSC with ECM gel from Engelbreth-Holm-Swarm murine sarcoma and a third cell culture medium comprising 3-10 mmol/l glucose, 5-15% FBS, 3-30 mmol/l nicotinamide, 5-50 ng/ml epidermal growth factor, 5-500 ng/ml of FGF-10, and 50-600 nmol/l (−)-indolactam V and incubating the at least one ESSC for about 5-15 days; then d) contacting the at least one ESSC with a fourth cell culture medium comprising 5-15% FBS, 1-100 nmol/l exendin-4, and 5-500 ng/ml Activin A and incubating the at least one ESSC for about 3-15 days; thereby deriving an insulin-producing cell from an ESSC.

In one embodiment, the ESSC is obtained from at least one biological sample selected from the group consisting of endometrium, endometrial stroma, endometrial membrane, and menstrual blood.

In one embodiment, the ESSC is a human ESSC.

In one embodiment, the first cell culture medium comprises 25 mmol/l glucose, 10% FBS and 10⁻⁶ mol/l retinoic acid, and the at least one ESSC is incubated in the first cell culture medium for about 24 hours.

In one embodiment, the second cell culture medium comprises 25 mmol/l glucose and 10% FBS and the at least one ESSC is incubated in the second cell culture medium for about 2 days.

In one embodiment, the third cell culture medium comprises 5.56 mmol/l glucose, 10% FBS, 10 mmol/l nicotinamide, 20 ng/ml epidermal growth factor, 50 ng/ml of FGF-10, and 300 nmol/l (−)-indolactam V and the at least one ESSC is incubated in the third cell culture medium for about 9 days.

In one embodiment, the fourth cell culture medium comprises 10% FBS, 10 nmol/l exendin-4, and 50 ng/ml Activin A and the at least one ESSC is incubated in the first cell culture medium for about 7 days.

The present invention also provides a composition comprising an insulin-producing cell derived from an ESSC by the methods of the invention.

In one embodiment, the composition comprising an insulin-producing cell derived from an ESSC is generated by a) contacting at least one ESSC with a first cell culture medium comprising 20-30 mmol/l glucose, 5-15% FBS and 10⁻⁵-10⁻⁷ mol/l retinoic acid, and incubating the at least one cell for about 12-36 hours; then b) contacting the at least one ESSC with a second cell culture medium comprising 20-30 mmol/l glucose and 5-15% FBS and incubating the at least one ESSC for about 1-4 days; then c) contacting the at least one ESSC with ECM gel from Engelbreth-Holm-Swarm murine sarcoma and a third cell culture medium comprising 3-10 mmol/l glucose, 5-15% FBS, 3-30 mmol/l nicotinamide, 5-50 ng/ml epidermal growth factor, 5-500 ng/ml of FGF-10, and 50-600 nmol/l (−)-indolactam V and incubating the at least one ESSC for about 5-15 days; then d) contacting the at least one ESSC with a fourth cell culture medium comprising 5-15% FBS, 1-100 nmol/l exendin-4, and 5-500 ng/ml Activin A and incubating the at least one ESSC for about 3-15 days.

In one embodiment, the ESSC is obtained from at least one biological sample selected from the group consisting of endometrium, endometrial stroma, endometrial membrane, and menstrual blood.

In one embodiment, the ESSC is a human ESSC.

In one embodiment, the insulin-producing cell exhibits at least one β cell marker selected from the group consisting of insulin, PAX4, PDX1, and GLUT2.

The invention also provides a method of treating a subject having diabetes comprising the steps of: administering at least one insulin-producing cell derived from an ESSC to the subject, wherein the insulin-producing cell secretes insulin within the subject, thereby treating the subject having diabetes.

In one embodiment, the ESSC is obtained from at least one biological sample selected from the group consisting of endometrium, endometrial stroma, endometrial membrane, and menstrual blood.

In one embodiment, the ESSC is a human ESSC.

In one embodiment, the ESSC is obtained from the subject.

In one embodiment, the diabetes is at least one selected from the group consisting of diabetes type I, diabetes type II and gestational diabetes.

In one embodiment, the at least one insulin-producing cell is administered by parenteral injection.

In one embodiment, the insulin-producing cell is derived from an ESSC according to the methods of the invention. For example, the insulin-producing cell is derived from an ESSC by: a) contacting at least one ESSC with a first cell culture medium comprising 20-30 mmol/l glucose, 5-15% FBS and 10⁻⁵-10⁻⁷ mol/l retinoic acid, and incubating the at least one cell for about 12-36 hours; then b) contacting the at least one ESSC with a second cell culture medium comprising 20-30 mmol/l glucose and 5-15% FBS and incubating the at least one ESSC for about 1-4 days; then c) contacting the at least one ESSC with ECM gel from Engelbreth-Holm-Swarm murine sarcoma and a third cell culture medium comprising 3-10 mmol/l glucose, 5-15% FBS, 3-30 mmol/l nicotinamide, 5-50 ng/ml epidermal growth factor, 5-500 ng/ml of FGF-10, and 50-600 nmol/l (−)-indolactam V and incubating the at least one ESSC for about 5-15 days; then d) contacting the at least one ESSC with a fourth cell culture medium comprising 5-15% FBS, 1-100 nmol/l exendin-4, and 5-500 ng/ml Activin A and incubating the at least one ESSC for about 3-15 days.

In one embodiment, the insulin-producing cell exhibits at least one β cell marker selected from the group consisting of insulin, PAX4, PDX1, and GLUT2.

The invention also provides a kit comprising at least one insulin-producing cell derived from an ESSC according to the methods of the invention. For example, the insulin-producing cell is derived from an ESSC by: a) contacting at least one ESSC with a first cell culture medium comprising 20-30 mmol/l glucose, 5-15% FBS and 10⁻⁵-10⁻⁷ mol/l retinoic acid, and incubating the at least one cell for about 12-36 hours; then b) contacting the at least one ESSC with a second cell culture medium comprising 20-30 mmol/l glucose and 5-15% FBS and incubating the at least one ESSC for about 1-4 days; then c) contacting the at least one ESSC with ECM gel from Engelbreth-Holm-Swarm murine sarcoma and a third cell culture medium comprising 3-10 mmol/l glucose, 5-15% FBS, 3-30 mmol/l nicotinamide, 5-50 ng/ml epidermal growth factor, 5-500 ng/ml of FGF-10, and 50-600 nmol/l (−)-indolactam V and incubating the at least one ESSC for about 5-15 days; then d) contacting the at least one ESSC with a fourth cell culture medium comprising 5-15% FBS, 1-100 nmol/l exendin-4, and 5-500 ng/ml Activin A and incubating the at least one ESSC for about 3-15 days.

In one embodiment, the insulin-producing cell exhibits at least one β cell marker selected from the group consisting of insulin, PAX4, PDX1, and GLUT2.

In one embodiment, the ESSC is obtained from at least one biological sample selected from the group consisting of endometrium, endometrial stroma, endometrial membrane, and menstrual blood.

In one embodiment, the ESSC is a human ESSC.

The invention provides a container comprising at least one insulin-producing cell derived from an ESSC according to the methods of the invention. For example, the insulin-producing cell is derived from an ESSC by: a) contacting at least one ESSC with a first cell culture medium comprising 20-30 mmol/l glucose, 5-15% FBS and 10⁻⁵-10⁻⁷ mol/l retinoic acid, and incubating the at least one cell for about 12-36 hours; then b) contacting the at least one ESSC with a second cell culture medium comprising 20-30 mmol/l glucose and 5-15% FBS and incubating the at least one ESSC for about 1-4 days; then c) contacting the at least one ESSC with ECM gel from Engelbreth-Holm-Swarm murine sarcoma and a third cell culture medium comprising 3-10 mmol/l glucose, 5-15% FBS, 3-30 mmol/l nicotinamide, 5-50 ng/ml epidermal growth factor, 5-500 ng/ml of FGF-10, and 50-600 nmol/l (−)-indolactam V and incubating the at least one ESSC for about 5-15 days; then d) contacting the at least one ESSC with a fourth cell culture medium comprising 5-15% FBS, 1-100 nmol/l exendin-4, and 5-500 ng/ml Activin A and incubating the at least one ESSC for about 3-15 days.

In one embodiment, the insulin-producing cell exhibits at least one β cell marker selected from the group consisting of insulin, PAX4, PDX1, and GLUT2.

In one embodiment, the ESSC is obtained from at least one biological sample selected from the group consisting of endometrium, endometrial stroma, endometrial membrane, and menstrual blood.

In one embodiment, the ESSC is a human ESSC.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A-1E, depicts the characterization of human endometrial stromal stem cell (ESSC) population by flow cytometry analysis. Human ESSC population after the second passage were subjected to flow cytometry analysis and compared to a negative control. FIG. 1A is a graph of flow cytometry analysis for CD45. FIG. 1B is a graph of flow cytometry analysis for CD45CD31. FIG. 1C is a graph of flow cytometry analysis for CD45PDGFβ-R. FIG. 1D is a graph of flow cytometry analysis for CD45CD 146. FIG. 1E is a graph of flow cytometry analysis for CD45CD90. APC, allophycocyanin; FITC, fluorescein isothiocyanate, PE, phycoerythrin.

FIG. 2, comprising FIGS. 2A-2G, depicts the differential expression of pan-pancreatic markers in treated ESSC in vitro. Successful ESSC induction was assessed by analysis of expression levels of genes that are associated with pancreatic β-cell maturation. FIG. 2A is a schematic representation depicting gene expression throughout pancreatic β-cell maturation. FIG. 2B is a photograph depicting human ESSC morphology in monolayer culture at the end of step one (original magnification×10), followed by detachment and replating on ECM gel in step two. FIG. 2C is a photograph depicting the cells in step two, which have formed three-dimensional clusters. FIG. 2D is a photograph depicting the end of step three, wherein further aggregation and clusters with islet-like morphology were observed. Scale Bar=30 μm. FIG. 2E is a graph depicting gene expression of differentiated cells which were harvested after step three. RNA was extracted and subjected to qRT-PCR. The amplification product is displayed by mass (ng) of cDNA (generated from a standard curve for each gene) and presented as the ratio of each gene to βAct. Mature pancreatic β-cell markers PDX1, PAX4, GLUT2, and INS were all increased in cells that underwent the differentiation treatment (left y-axis). Early development genes were similarly assessed; HNF6α and NGN3 were not significantly increased by the differentiation treatment (right y-axis), demonstrating a preponderance of only those genes marking mature pancreatic β-like cells. FIG. 2F is a photograph of a gel depicting the expression of PDX-1, INS, and βAct, which were further assessed at the end of each step using standard PCR and gel electrophoresis. Analysis of the resulting gel showed pronounced insulin expression by the end of step three. FIG. 2G is a PDX-1 was expressed through all the three stages. Gene expression in pancreatic islet cells is shown as a positive control (*Indicates P<0.001 treated versus untreated cells). Integration of the small molecule ILV in step two resulted with higher expression levels of PDX1 and NGN3. cDNA, complementary DNA; ECM, extracellular matrix, ILV, indolactam V, qRT-PCR, quantitative reverse transcriptase, PCR.

FIG. 3, comprising FIGS. 3A-3B, depicts the increased production and secretion of insulin by treated cells in vitro. FIG. 3A is a series of photographs depicting insulin production by undifferentiated (upper panel) and differentiated (lower panel) clustered cell formations in vitro. By the end of step three, cultured cells were washed three times, fixed with 4% paraformaldehyde, and immunofluorescence was used to detect insulin (GFP). Cells were also stained with DAPI. Scale Bar=30 μm. DAPI, 4′,6-diamidino-2-phenylindole; ELISA, enzyme-linked immunosorbent assay; GFP, green fluorescent protein. FIG. 3B is a graph depicting insulin secretion from differentiated and undifferentiated cultures was assessed using ELISA. To determine whether insulin secretion was glucose dependent, cells were treated with either 5 mmol/l or 25 mmol/l glucose. Differentiated cells secreted insulin in response to glucose stimulation. Undifferentiated cells failed to produce measurable insulin even after a glucose challenge (undifferentiated versus differentiated cells, *P<0.05; for 5 mmol/l versus 25 mmol/l differentiated cells, **P<0.01).

FIG. 4, comprising FIGS. 4A-4C, depicts the production and secretion of insulin by differentiated human ESSC in a murine diabetes model. FIG. 4A is a graph depicting how SCID mice were treated with STZ to induce diabetes and hyperglycemia confirmed. Differentiated ESSCs=black circles (N=8) or undifferentiated ESSCs=“X” (N=8) were transplanted under the kidney capsule of diabetic mice. Untreated nondiabetic SCID mice were used as a second control=black squares (N=8). Weekly measurements of blood glucose were obtained for 5 weeks after surgery. FIG. 4B is a graph depicting glucose levels of diabetic mice that were transplanted with the xenograft containing differentiated pancreatic β-like cells stabilized during the weeks following the procedure. Mice that were transplanted with undifferentiated cells developed a more severe hyperglycemia in the weeks following the transplant. As expected, the nondiabetic untreated (UT) mice maintained normal glucose levels. (Error bars are SEM, each time point after transplant P<0.05 between each group). Below, the same data were analyzed by calculating the ratio between the mean weekly measured values at each time point and the mean value from time zero (time of transplant). The control mice transplanted with undifferentiated cells showed increasing glucose levels consistent with worsening diabetic control. The animals transplanted with the xenograft containing the differentiated β-like cells showed no further increase in glucose levels, similar to the nondiabetic untreated controls (Error bars are SEM, each time point P<0.05 xenograft versus both diabetic control and undifferentiated transplant). FIG. 4C is a series of images depicting members of each group (transplanted with differentiated and undifferentiated ESSCs). Members were sacrificed and kidneys containing the xenograft were subjected to IHC-IF (kidney labeled as K, xenograft labeled as Xe). In the control no insulin signal was observed (upper panel, original magnification×40). Insulin secretion was observed in the group that was transplanted with differentiated ESSCs within the transplant xenograft (middle panel, original magnification×40; lower panel, original magnification×63). Scale bars for (original magnification×40) and (original magnification×63) are 20 and 6 μm, respectively. IHC-IF, immunohistochemistry-immunofluorescence; SCID, severe combined immunodeficiency, STZ, streptozotocin.

FIG. 5, comprising FIGS. 5A-5C, depicts the results of experiments demonstrating that mice transplanted with differentiated β-like cells show no evidence of diabetic complications. FIG. 5A is a photograph of a mouse transplanted with differentiated ESSCs. Four weeks after transplanting the xenograft, the two groups of mice (injected with treated ESSCs and injected with undifferentiated ESSCs) were evaluated for complications associated with diabetes. FIG. 5B is a photograph of a mouse transplanted with undifferentiated cells. Unlike the mice that were transplanted with differentiated ESSCs that formed insulin secreting β-like cells, the mice that were transplanted with the undifferentiated cells demonstrated a large number of complications and symptoms typical of diabetes; these included cataract, signs of dehydration, appearance of a hump, loss of fur brightness, and a more passive behavior. FIG. 5C is a graph depicting the weights of each member of the three groups (transplanted with differentiated cells or transplanted with undifferentiated, and untreated nondiabetic mice) measured and analyzed by calculating the fold change from values in week 0. Weights of the mice transplanted with differentiated cells (0.99) were identical to those of the wild type nondiabetic mice (1.00), while those that were transplanted with undifferentiated cells underwent an 8% weight loss (0.92) in this time period (*Indicate P<0.05 compared to untreated control).

### DETAILED DESCRIPTION

The invention relates to the discovery that endometrial stromal stem cells (ESSC) can be differentiated into progeny cells that produce insulin and express β cell markers. Thus, the invention includes the progeny of ESSC, including any cell type generated during the differentiation of ESSC towards cells that produce insulin and exhibit cell markers characteristic of insulin producing cells. In one embodiment, the invention includes a method of making an insulin-producing cell derived from an ESSC. In another embodiment, the invention includes a culture system for deriving an insulin-producing cell from an ESSC. In various embodiments, the invention includes a method of using the insulin-producing cell derived from an ESSC to treat a subject having a disease or disorder. In various embodiments, the disease or disorder treatable by the methods of the invention includes, but is not limited to, diabetes, including diabetes type I, diabetes type II and gestational diabetes.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, “autologous” refers to a biological material derived from the same individual into whom the material will later be re-introduced.

As used herein, “allogeneic” refers to a biological material derived from a genetically different individual of the same species as the individual into whom the material will be introduced.

As used herein, the term “basal medium” refers to a solution of amino acids, vitamins, salts, and nutrients that is effective to support the growth of cells in culture, although normally these compounds will not support cell growth unless supplemented with additional compounds. The nutrients include a carbon source (e.g., a sugar such as glucose) that can be metabolized by the cells, as well as other compounds necessary for the cells' survival. These are compounds that the cells themselves cannot synthesize, due to the absence of one or more of the gene(s) that encode the protein(s) necessary to synthesize the compound (e.g., essential amino acids) or, with respect to compounds which the cells can synthesize, because of their particular developmental state the gene(s) encoding the necessary biosynthetic proteins are not being expressed as sufficient levels. A number of base media are known in the art of mammalian cell culture, such as Dulbecco's Modified Eagle Media (DMEM), Knockout-DMEM (KO-DMEM), and DMEM/F12, although any base medium that supports the growth of primate embryonic stem cells in a substantially undifferentiated state can be employed.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

The term “cell medium” as used herein, refers to a medium useful for culturing cells. An example of a cell medium is a medium comprising DMEM/F 12 Ham's, 10% fetal bovine serum, 100 U penicillin/100 mg streptomycin/0.25 μg Fungizone. Typically, the cell medium comprises a base medium, serum and an antibiotic/antimycotic. However, cells can be cultured with stromal cell medium without an antibiotic/antimycotic and supplemented with at least one growth factor. Preferably the growth factor is human epidermal growth factor (hEGF). The preferred concentration of hEGF is about 1-50 ng/ml, more preferably the concentration is about 5 ng/ml. The preferred base medium is DMEM/F12 (1:1). The preferred serum is fetal bovine serum (FBS) but other sera may be used including horse serum or human serum. Preferably up to 20% FBS will be added to the above media in order to support the growth of stromal cells. However, a defined medium could be used if the necessary growth factors, cytokines, and hormones in FBS for cell growth are identified and provided at appropriate concentrations in the growth medium. It is further recognized that additional components may be added to the culture medium. Such components include but are not limited to antibiotics, antimycotics, albumin, growth factors, amino acids, and other components known to the art for the culture of cells. Antibiotics which can be added into the medium include, but are not limited to, penicillin and streptomycin. The concentration of penicillin in the culture medium is about 10 to about 200 units per ml. The concentration of streptomycin in the culture medium is about 10 to about 200 μg/ml. However, the invention should in no way be construed to be limited to any one medium for culturing cells. Rather, any media capable of supporting cells in tissue culture may be used.

The term “differentiated cell” refers to a cell of a more specialized cell type derived from a cell of a less specialized cell type (e.g., a stem cell or ESSC) in a cellular differentiation process.

“Differentiation medium” is used herein to refer to a cell growth medium comprising an additive or a lack of an additive such that a stem cell, ESSC or other such progenitor cell, that is not fully differentiated, develops into a cell with some or all of the characteristics of a differentiated cell when incubated in the medium.

A “donor” is a subject used as a source of a biological material containing ESSC, such as endometrium, endometrial stroma, endometrial membrane, or menstrual blood. A “recipient” is a subject which accepts a biological material, such as, by way of examples, an ESSC or differentiated progeny of an ESSC. In autologous transfers, the donor and recipient are one and the same, i.e., syngeneic.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

As used herein, a “cell culture” refers to the maintenance or growth of one or more cells in vitro or ex vivo. Thus, for example, an ESSC culture is one or more cells in a growth medium of some kind. A “culture medium” or “growth medium” are used interchangeably herein to mean any substance or preparation used for sustaining or maintaining cells.

An “effective amount” or “therapeutically effective amount” of a compound is that amount of compound which is sufficient to provide a beneficial effect to the subject to which the compound is administered.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

An “isolated cell” refers to a cell which has been separated from other components and/or cells which naturally accompany the isolated cell in a tissue or mammal.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “polynucleotide” means a single strand or parallel and anti-parallel strands of a nucleic acid. Thus, a polynucleotide may be either a single-stranded or a double-stranded nucleic acid. The term “nucleic acid” typically refers to large polynucleotides. The terms “nucleic acid” and “polynucleotide” and the like refer to at least two or more ribo- or deoxy-ribonucleic acid base pairs (nucleotides) that are linked through a phosphoester bond or equivalent. Nucleic acids include polynucleotides and polynucleosides. Nucleic acids include single, double or triplex, circular or linear, molecules. Exemplary nucleic acids include RNA, DNA, cDNA, genomic nucleic acid, naturally occurring and non naturally occurring nucleic acid, e.g., synthetic nucleic acid.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A “recombinant polypeptide” is one which is produced upon expression of a recombinant polynucleotide.

The term “transfected” when use in reference to a cell (e.g. a host pluripotent stem cell), means a genetic change in a cell following incorporation of an exogenous molecule, for example, a nucleic acid (e.g., a transgene) or protein into the cell. Thus, a “transfected” cell is a cell into which, or a progeny thereof in which an exogenous molecule has been introduced by the hand of man, for example, by recombinant DNA techniques.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

As used herein, the term “transgene” means an exogenous nucleic acid sequence which exogenous nucleic acid is encoded by a transgenic cell or mammal.

A “recombinant cell” is a cell that comprises a transgene. Such a cell may be a eukaryotic cell or a prokaryotic cell.

By the term “exogenous nucleic acid” is meant that the nucleic acid has been introduced into a cell or an animal using technology which has been developed for the purpose of facilitating the introduction of a nucleic acid into a cell or an animal.

As used herein, a “substantially purified” cell is a cell that is essentially free of other cell types. Thus, a substantially purified cell refers to a cell which has been purified from other cell types with which it is normally associated in its naturally-occurring state.

A “therapeutic” treatment is a treatment administered to a subject who exhibits a sign or symptom of pathology, for the purpose of diminishing or eliminating those signs or symptoms.

As used herein, “treating a disease or disorder” means reducing the frequency or severity with which a sign or symptom of the disease or disorder is experienced by a patient.

The phrase “therapeutically effective amount,” as used herein, refers to an amount that is sufficient or effective to prevent or treat (delay or prevent the onset of, prevent the progression of, inhibit, decrease or reverse) a disease or disorder.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The invention relates to the discovery that an ESSC can be differentiated to produce an insulin-producing cell and exhibiting at least one β cell marker. Thus, the invention relates to culture systems for making an insulin-producing cell derived from ESSC, methods of making an insulin-producing cell derived from an ESSC, and methods of using the insulin-producing cell derived from an ESSC to treat a subject having a disease or disorder, such as diabetes, including diabetes type I, diabetes type II and gestational diabetes.

The invention provides, among other things, mammalian (e.g., human) ESSC, populations and pluralities of mammalian ESSC, cultured populations and pluralities of mammalian ESSC, and differentiated progeny of ESSC. Such ESSC are characterized by various features, including, for example, the presence or absence of various phenotypic markers, the ability to undergo cell division within a given time period in a suitable growth medium, the ability to produce certain proteins, and a characteristic morphology. In one embodiment, ESSC express a marker selected from PDFGFβ-R, CD146, and CD90. In a further embodiment, ESSC do not express a marker selected from CD45 or CD31. Non-limiting exemplary cell medium are a liquid medium such as DMEM, alpha-MEM or RPMI. Other suitable medium for ESSC cell maintenance, growth and proliferation would be known to the skilled artisan. Such media can include one or more of supplements, such as albumin, essential amino acids, non-essential amino acids, L-glutamine, a thyroid hormone, vitamins, etc.

The invention therefore also provides cells differentiated with respect to mammalian ESSC, wherein the cells are the progeny of a mammalian ESSC. A “progeny” of an ESSC refers to any and all cells derived from ESSC as a result of clonal proliferation or differentiation. As used herein, a “progenitor cell” is a parent cell committed to give rise to a distinct cell lineage by a series of cell divisions. Specific progenitor cell types may sometimes be identified by markers. A “precursor cell” refers to a cell from which another cell is formed. It encompasses a cell that precedes the existence of a later, more developmentally mature cell. In contrast to the maturation of progenitor cells, which is marked by cell division, the developmental maturation of a precursor cell may include any number of processes or events, including, but not limited to, differential gene expression, or change in size, morphology, or location. As used herein, both progenitor and precursor cells are progeny of and distinct from a pluripotent stem cell. A “developmental intermediate” cell refers to any cell that is either a progenitor or precursor cell that is distinct from the pluripotent stem cells and the ultimately differentiated cell type.

ESSC of the invention include ESSC populations and pluralities of ESSC (and progeny thereof), and cultures of ESSC (cell cultures, and progeny cultures). A population or plurality or culture of ESSC (or progeny thereof) means that there are a collection of such cells. In various embodiments, ESSC population, plurality of ESSC or culture of ESSC (or progeny thereof) include mammalian ESSC that represent at least 25%, 50%, 75%, 90% or more of the total number of cells in the population or plurality or culture.

In a population or plurality of ESSC, or in a culture of ESSC, a majority of cells, but not all cells present may or may not express a particular phenotypic marker indicative of an ESSC. Such cells are typically present in the population, plurality or culture at a smaller percentage of the total number of ESSC present. In various embodiments, an ESSC population, plurality of ESSC or culture of ESSC include cells in which greater than about 50%, 60%, 70%, 80%, 90%-95% or more (e.g., 96%, 97%, 98%, etc. . . . 100%) of the cells express a particular phenotypic marker. In particular aspects, 75%, 80%, 85%, 90%, 95% or more of the population, plurality of pluripotent stem cells or culture of pluripotent stem cells express a marker selected from PDFGFβ-R, CD146, and CD90. In various embodiments, an ESSC population, plurality of ESSC or culture of ESSC include cells in which less than about 25%, 20%, 15%, 10%, 5% or less (e.g., 4%, 4%, 2%, 1%) of the cells express a particular phenotypic marker. In various aspects, in a population of ESSC, plurality of ESSC or a culture of ESSC, 25%, 20%, 15%, 10%, 5% or less (e.g., 4%, 3%, 2%, 1%) of the cells express a marker selected from CD34 and CD31.

ESSC cells of the invention (or progeny thereof) include co-cultures and mixed populations. Such co-cultures and mixed cell populations of cells include a first mammalian (e.g., a human ESSC) cell, and a second cell distinct from the first cell. A second cell can comprise a population of cells. Non-limiting examples of exemplary cells distinct from mammalian (e.g., a human ESSC) cell include a cell, T cell, dendritic cell, NK cell, monocyte, macrophage or PBMCs. Additional non-limiting examples of exemplary cells distinct from mammalian (e.g., a human ESSC) cell include different adult or embryonic stem cells; totipotent, pluripotent or multipotent stem cell or progenitor or precursor cells; cord blood stem cells; placental stem cells; bone marrow stem cells; amniotic fluid stem cells; neuronal stem cells; circulating peripheral blood stem cells; mesenchymal stem cells; germinal stem cells; adipose tissue derived stem cells; exfoliated teeth derived stem cells; hair follicle stem cells; dermal stem cells; parthenogenically derived stem cells; reprogrammed stem cells; side population stem cells; and differentiated cells.

The presence or absence of a given phenotypic marker can be determined using the methods disclosed elsewhere herein. Thus, the presence or absence of a given phenotypic marker can be determined by an antibody that binds to the marker. Accordingly, marker expression can be determined by an antibody that binds to each of the respective markers, such as PDFGFβ-R, CD146, and CD90, etc., in order to indicate which or how many ESSC are present in a given population, plurality or culture of ESSC express the marker. Additional methods of detecting these and other phenotypic markers are known to one of skill in the art.

Cell cultures of ESSC can take on a variety of formats. For instance, an “adherent culture” refers to a culture in which cells in contact with a suitable growth medium are present, and can be viable or proliferate while adhered to a substrate. Likewise, a “continuous flow culture” refers to the cultivation of cells in a continuous flow of fresh medium to maintain cell viability, e.g. growth.

In one embodiment, the invention includes a culture system comprising at least one insulin-producing cell derived from an ESSC. In various embodiments described elsewhere herein, the invention includes a method of using the ESSC-derived insulin-producing cell culture system of the invention to conduct insulin-producing cell differentiation analyses, to screen for and identify modulators of insulin-producing cell differentiation, and to monitor the effect of modulators of insulin-producing cell differentiation.

In one embodiment, the culture system of the invention comprises at least one ESSC-derived insulin-producing cell cultured in a suitable media. One non-limiting example of a suitable media is DMEM/F12 comprising 10% FBS.

The culture system of the invention can include any kind of substrate, surface, scaffold or container known in the art useful for culturing cells. Non-limiting examples of such containers include cell culture plates, dishes and flasks. Other suitable substrates, surfaces and containers are described in Culture of Animal Cells: a manual of basic techniques (3rd edition), 1994, R. I. Freshney (ed.), Wiley-Liss, Inc.; Cells: a laboratory manual (vol. 1), 1998, D. L. Spector, R. D. Goldman, L. A. Leinwand (eds.), Cold Spring Harbor Laboratory Press; Embryonic Stem Cells, 2007, J. R. Masters, B. O. Palsson and J. A. Thomson (eds.), Springer; Stem Cell Culture, 2008, J. P. Mather (ed.) Elsevier; and Animal Cells: culture and media, 1994, D. C. Darling, S. J. Morgan John Wiley and Sons, Ltd. In some embodiments, the culture system comprises a two-dimensional scaffold. In other embodiments, the culture system comprises a three-dimensional scaffold.

Mammalian ESSC and their progeny include individual cells, and populations and pluralities of cells, that are isolated or purified. As used herein, the terms “isolated” or “purified” refers to made or altered “by the hand of man” from the natural state (i.e., when it has been removed or separated from one or more components of the original natural in vivo environment.) An isolated composition can but need not be substantially separated from other biological components of the organism in which the composition naturally occurs. An example of an isolated cell would be an ESSC obtained from a subject such as a human. “Isolated” also refers to a composition, for example, an ESSC separated from one or more contaminants (i.e. materials and substances that differ from the cell). A population, plurality or culture of ESSC (or their progeny) is typically substantially free of cells and materials with which it is be associated in nature. The term “purified” refers to a composition free of many, most or all of the materials with which it typically associates with in nature. Thus, an ESSC or its progeny is considered to be substantially purified when separated from other tissue components. Purified therefore does not require absolute purity. Furthermore, a “purified” composition can be combined with one or more other molecules. Thus, the term “purified” does not exclude combinations of compositions. Purified can be at least about 50%, 60% or more by numbers or by mass. Purity can also be about 70% or 80% or more, and can be greater, for example, 90% or more. Purity can be less, for example, in a pharmaceutical carrier the amount of a cells or molecule by weight % can be less than 50% or 60% of the mass by weight, but the relative proportion of the cells or molecule compared to other components with which it is normally associated with in nature will be greater. Purity of a population or composition of cells can be assessed by appropriate methods that would be known to the skilled artisan.

A primary isolate of an ESSC of the invention can originate from or be derived from endometrium, endometrial stroma, endometrial membrane, or menstrual blood. Progeny of primary isolate ESSC, which include all descendents of the first, second, third and any and all subsequent generations and cells taken or obtained from a primary isolate, that maintain sternness (e.g., phenotypic marker expression profile, doubling time, morphology, secretion of proteins, etc.) can be obtained from a primary isolate or subsequent expansion of a primary isolate. Subsequent expansion results in progeny of ESSC that can in turn comprise the populations or pluralities of ESSC, the cultures of ESSC, progeny of ESSC, co-cultures, etc. Thus, ESSC of the invention refers to a cell from a primary isolate from endometrium, endometrial stroma, endometrial membrane, or menstrual blood, and any progeny cell therefrom. The term “derived” or “originates,” when used in reference to an ESSC therefore means that the cells or parental cells of any previous generation at one point in time originated from endometrium, endometrial stroma, endometrial membrane, or menstrual blood. Accordingly, ESSC are not limited to those from a primary isolate, but can be any subsequent progeny thereof or any subsequent doubling of the progeny thereof provided that the cell has the desired phenotypic markers, doubling time, or any other characteristic feature set forth herein.

### Genetic Modification

In the context of gene therapy, the cells of the invention can be treated with a gene of interest prior to delivery of the cells into the recipient. In some cases, such cell-based gene delivery can present significant advantages of other means of gene delivery, such as direct injection of an adenoviral gene delivery vector. Delivery of a therapeutic gene that has been pre-inserted into cells avoids the problems associated with penetration of gene therapy vectors into desired cells in the recipient.

Accordingly, the invention provides the use of genetically modified cells that have been cultured according to the methods of the invention. Genetic modification may, for instance, result in the expression of exogenous genes (“transgenes”) or in a change of expression of an endogenous gene. Such genetic modification may have therapeutic benefit. Alternatively, the genetic modification may provide a means to track or identify the cells so-modified, for instance, after implantation of a composition of the invention into an individual. Tracking a cell may include tracking migration, assimilation and survival of a transplanted genetically-modified cell. Genetic modification may also include at least a second gene. A second gene may encode, for instance, a selectable antibiotic-resistance gene or another selectable marker.

In some embodiment, mammalian ESSC (and their progeny) include those transfected with a nucleic acid. Such nucleic acids can encode proteins, polypeptides and peptides, for example, proteins, polypeptides and peptides to substitute for defectiveness, absence or deficiency of endogenous protein, polypeptide or peptide in a subject.

The cells of the invention may be genetically modified using any method known to the skilled artisan. See, for instance, Sambrook et al. (2001, Molecular Cloning: A Laboratory Manual, Cold Spring Harbor Laboratory Press, Cold Spring Harbor, N.Y.), and in Ausubel et al., Eds, (1997, Current Protocols in Molecular Biology, John Wiley & Sons, New York, N.Y.). For example, a cell may be exposed to an expression vector comprising a nucleic acid including a transgene, such that the nucleic acid is introduced into the cell under conditions appropriate for the transgene to be expressed within the cell. The transgene generally is an expression cassette, including a polynucleotide operably linked to a suitable promoter. The polynucleotide can encode a protein, or it can encode biologically active RNA (e.g., antisense RNA or a ribozyme). Thus, for example, the polynucleotide can encode a gene conferring resistance to a toxin, a hormone (such as peptide growth hormones, hormone releasing factors, sex hormones, adrenocorticotrophic hormones, cytokines (e.g., interferins, interleukins, lymphokines), etc.), a cell-surface-bound intracellular signaling moiety (e.g., cell adhesion molecules, hormone receptors, etc.), a factor promoting a given lineage of differentiation (e.g., bone morphogenic protein (BMP)), etc., and insulin.

Nucleic acids can be of various lengths. Nucleic acid lengths typically range from about 20 nucleotides to 20 Kb, or any numerical value or range within or encompassing such lengths, 10 nucleotides to 10 Kb, 1 to 5 Kb or less, 1000 to about 500 nucleotides or less in length. Nucleic acids can also be shorter, for example, 100 to about 500 nucleotides, or from about 12 to 25, 25 to 50, 50 to 100, 100 to 250, or about 250 to 500 nucleotides in length, or any numerical value or range or value within or encompassing such lengths. Shorter polynucleotides are commonly referred to as “oligonucleotides” or “probes” of single- or double-stranded DNA.

Nucleic acids can be produced using various standard cloning and chemical synthesis techniques. Techniques include, but are not limited to nucleic acid amplification, e.g., polymerase chain reaction (PCR), with genomic DNA or cDNA targets using primers (e.g., a degenerate primer mixture) capable of annealing to antibody encoding sequence. Nucleic acids can also be produced by chemical synthesis (e.g., solid phase phosphoramidite synthesis) or transcription from a gene. The sequences produced can then be translated in vitro, or cloned into a plasmid and propagated and then expressed in a cell (e.g., a host cell such as yeast or bacteria, a eukaryote such as an animal or mammalian cell or in a plant).

Nucleic acids can be included within vectors as cell transfection typically employs a vector. The term “vector,” refers to, e.g., a plasmid, virus, such as a viral vector, or other vehicle known in the art that can be manipulated by insertion or incorporation of a polynucleotide, for genetic manipulation (i.e., “cloning vectors”), or can be used to transcribe or translate the inserted polynucleotide (i.e., “expression vectors”). Such vectors are useful for introducing polynucleotides in operable linkage with a nucleic acid, and expressing the transcribed encoded protein in cells in vitro, ex vivo or in vivo.

A vector generally contains at least an origin of replication for propagation in a cell. Control elements, including expression control elements, present within a vector, are included to facilitate transcription and translation. The term “control element” is intended to include, at a minimum, one or more components whose presence can influence expression, and can include components other than or in addition to promoters or enhancers, for example, leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of a gene of interest, stop codons, among others.

Vectors included are those based on viral vectors, such as retroviral (lentivirus for infecting dividing as well as non-dividing cells), foamy viruses (U.S. Pat. Nos. 5,624,820, 5,693,508, 5,665,577, 6,013,516 and 5,674,703; WO92/05266 and WO92/14829), adenovirus (U.S. Pat. Nos. 5,700,470, 5,731,172 and 5,928,944), adeno-associated virus (AAV) (U.S. Pat. No. 5,604,090), herpes simplex virus vectors (U.S. Pat. No. 5,501,979), cytomegalovirus (CMV) based vectors (U.S. Pat. No. 5,561,063), reovirus, rotavirus genomes, simian virus 40 (SV40) or papilloma virus (Cone et al., Proc. Natl. Acad. Sci. USA 81:6349 (1984); Eukaryotic Viral Vectors, Cold Spring Harbor Laboratory, Gluzman ed., 1982; Sarver et al., Mol. Cell. Biol. 1:486 (1981); U.S. Pat. No. 5,719,054). Adenovirus efficiently infects slowly replicating and/or terminally differentiated cells and can be used to target slowly replicating and/or terminally differentiated cells. Simian virus 40 (SV40) and bovine papilloma virus (BPV) have the ability to replicate as extra-chromosomal elements (Eukaryotic Viral Vectors, Cold Spring Harbor Laboratory, Gluzman ed., 1982; Sarver et al., Mol. Cell. Biol. 1:486 (1981)). Additional viral vectors useful for expression include reovirus, parvovirus, Norwalk virus, coronaviruses, paramyxo- and rhabdoviruses, togavirus (e.g., sindbis virus and semliki forest virus) and vesicular stomatitis virus (VSV) for introducing and directing expression of a polynucleotide or transgene in pluripotent stem cells or progeny thereof (e.g., differentiated cells).

Vectors including a nucleic acid can be expressed when the nucleic acid is operably linked to an expression control element. As used herein, the term “operably linked” refers to a physical or a functional relationship between the elements referred to that permit them to operate in their intended fashion. Thus, an expression control element “operably linked” to a nucleic acid means that the control element modulates nucleic acid transcription and as appropriate, translation of the transcript.

The term “expression control element” refers to nucleic acid that influences expression of an operably linked nucleic acid. Promoters and enhancers are particular non-limiting examples of expression control elements. A “promoter sequence” is a DNA regulatory region capable of initiating transcription of a downstream (3′ direction) sequence. The promoter sequence includes nucleotides that facilitate transcription initiation. Enhancers also regulate gene expression, but can function at a distance from the transcription start site of the gene to which it is operably linked. Enhancers function at either 5′ or 3′ ends of the gene, as well as within the gene (e.g., in introns or coding sequences). Additional expression control elements include leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of interest, and stop codons.

Expression control elements include “constitutive” elements in which transcription of an operably linked nucleic acid occurs without the presence of a signal or stimuli. For expression in mammalian cells, constitutive promoters of viral or other origins may be used. For example, SV40, or viral long terminal repeats (LTRs) and the like, or inducible promoters derived from the genome of mammalian cells (e.g., metallothionein IIA promoter; heat shock promoter, steroid/thyroid hormone/retinoic acid response elements) or from mammalian viruses (e.g., the adenovirus late promoter; mouse mammary tumor virus LTR) are used.

Expression control elements that confer expression in response to a signal or stimuli, which either increase or decrease expression of operably linked nucleic acid, are “regulatable.” A regulatable element that increases expression of operably linked nucleic acid in response to a signal or stimuli is referred to as an “inducible element.” A regulatable element that decreases expression of the operably linked nucleic acid in response to a signal or stimuli is referred to as a “repressible element” (i.e., the signal decreases expression; when the signal is removed or absent, expression is increased).

Expression control elements include elements active in a particular tissue or cell type, referred to as “tissue-specific expression control elements.” Tissue-specific expression control elements are typically more active in specific cell or tissue types because they are recognized by transcriptional activator proteins, or other transcription regulators active in the specific cell or tissue type, as compared to other cell or tissue types.

In accordance with the invention, there are provided ESSC and their progeny transfected with a nucleic acid or vector. Such transfected cells include but are not limited to a primary cell isolate, populations or pluralities of pluripotent stem cells, cell cultures (e.g., passaged, established or immortalized cell line), as well as progeny cells thereof (e.g., a progeny of a transfected cell that is clonal with respect to the parent cell, or has acquired a marker or other characteristic of differentiation).

The nucleic acid or protein can be stably or transiently transfected (expressed) in the cell and progeny thereof. The cell(s) can be propagated and the introduced nucleic acid transcribed and protein expressed. A progeny of a transfected cell may not be identical to the parent cell, since there may be mutations that occur during replication.

Viral and non-viral vector means of delivery into ESSC, in vitro, in vivo and ex vivo are included. Introduction of compositions (e.g., nucleic acid and protein) into target cells (e.g., host pluripotent stem cells) can be carried out by methods known in the art, such as osmotic shock (e.g., calcium phosphate), electroporation, microinjection, cell fusion, etc. Introduction of nucleic acid and polypeptide in vitro, ex vivo and in vivo can also be accomplished using other techniques. For example, a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers. A nucleic acid can be entrapped in microcapsules prepared by coacervation techniques or by interfacial polymerization, for example, by the use of hydroxymethylcellulose or gelatin-microcapsules, or poly(methylmethacrylate) microcapsules, respectively, or in a colloid system. Colloidal dispersion systems include macromolecule complexes, nano-capsules, microspheres, beads, and lipid-based systems, including oil-in-water emulsions, micelles, mixed micelles, and liposomes.

Liposomes for introducing various compositions into cells are known in the art and include, for example, phosphatidylcholine, phosphatidylserine, lipofectin and DOTAP (e.g., U.S. Pat. Nos. 4,844,904, 5,000,959, 4,863,740, and 4,975,282; and GIBCO-BRL, Gaithersburg, Md.). Piperazine based amphilic cationic lipids useful for gene therapy also are known (see, e.g., U.S. Pat. No. 5,861,397). Cationic lipid systems also are known (see, e.g., U.S. Pat. No. 5,459,127). Polymeric substances, microcapsules and colloidal dispersion systems such as liposomes are collectively referred to herein as “vesicles.”

### Methods

ESSC of the invention and their progeny can be sterile, and maintained in a sterile environment. Such ESSC, pluralities, populations, and cultures thereof can also be included in a medium, such as a liquid medium suitable for administration to a subject (e.g., a mammal such as a human).

Methods for producing a ESSC and their differentiated progeny are provided herein. In one embodiment, a method includes obtaining a tissue or blood sample, cloning one or more cells from the sample, selecting one or more cells based upon morphology or growth rate or phenotypic marker expression profile, thereby isolating an ESSC.

Methods for producing ESSC populations and pluralities of ESSC are also provided. In such methods, expanding ESSC for a desired number of cell divisions (doublings) thereby producing increased numbers or a population or plurality of ESSC. Relative proportions or amounts of ESSC within cell cultures include 50%, 60%, 70%, 80%, 90% or more ESSC in a population or plurality of cells.

Methods for producing a differentiated progeny cell of an ESSC (e.g., a progenitor cell, a precursor cell, a developmental intermediate, a differentiated cell, an insulin-producing cells) from ESSC are also provided.

In one embodiment, the invention includes an insulin-producing cell derived from an ESSC. In one embodiment, the invention includes a method of making an insulin-producing cell derived from an ESSC.

In various embodiments, the method of making an insulin-producing cell derived from an ESSC comprises a multi-step method of exposing at least one ESSC to series of chemicals over about a 12-30 day period. In various embodiments, prior to differentiation to an insulin-producing cell, ESSC are cultured in a suitable cell culture medium. One non-limiting example of a suitable cell culture medium is DMEM/F12 with 10% FBS. For Step 1 of the method of making an insulin-producing cell derived from an ESSC, an ESSC monolayer is bathed for about 12-36 hours in media comprising high glucose Dulbecco's modified Eagle medium (DMEM) 20-30 mmol/l, 5-15% FBS and 10⁻⁵-10⁻⁷ mol/l retinoic acid, and then bathed in media containing high glucose DMEM and 5-15% FBS for about 1-4 days. For Step 2, the cells are detached with 0.05% trypsin, seeded onto plates pre-coated with ECM gel from Engelbreth-Holm-Swarm murine sarcoma, and bathed in medium comprising low glucose DMEM 3-10 mmol/l, 5-15% FBS, 3-30 mmol/l nicotinamide, 5-50 ng/ml epidermal growth factor, 5-500 ng/ml of FGF-10, and 50-600 nmol/l of (−)-indolactam V for about 5-15 days. For Step 3, the cells are bathed in media comprising L-DMEM, 5-15% FBS, 1-100 nmol/l exendin-4, and 5-500 ng/ml Activin A for about 3-15 days. Optionally, the media described above can be supplemented with 1% penicillin/streptomycin and/or 1% amphotericin B. The skilled artisan will understand that many ESSC supplements are known in the art that can be used in the media described herein. Numerous types of serum, alone or in combination, may be used including human, fetal calf serum, or cord blood serum.

In a particular embodiment, prior to differentiation to an insulin-producing cell, ESSC are cultured in a suitable cell culture medium. One non-limiting example of a suitable cell culture medium is DMEM/F12 with 10% FBS. For Step 1 of the method of making an insulin-producing cell derived from an ESSC, an ESSC monolayer is bathed for about 24 hours in media comprising high glucose Dulbecco's modified Eagle medium (DMEM) 25 mmol/l, 10% FBS and 10⁻⁶ mol/l retinoic acid, and then bathed in media containing high glucose DMEM and 10% FBS for about 2 days. For Step 2, the cells are detached with 0.05% trypsin, seeded onto plates pre-coated with ECM gel from Engelbreth-Holm-Swarm murine sarcoma, and bathed in medium comprising low glucose DMEM 5.56 mmol/l, 10% FBS, 10 mmol/l nicotinamide, 20 ng/ml epidermal growth factor, 50 ng/ml of FGF-10, and 300 nmol/l of (−)-indolactam V for about 9 days. For Step 3, the cells are bathed in media comprising L-DMEM, 10% FBS, 10 nmol/l exendin-4, and 50 ng/ml Activin A for about 7 days. Optionally, the media described above can be supplemented with 1% penicillin/streptomycin and/or 1% amphotericin B. The skilled artisan will understand that many ESSC supplements are known in the art that can be used in the media described herein. Numerous types of serum, alone or in combination, may be used including human, fetal calf serum, or cord blood serum.

The quality of the insulin-producing cells derived from ESSC may be detected morphologically, by the ability of differentiated cells to self-assemble to form three-dimensional islet cell-like clusters, as well as expression of cell differentiation-related transcripts detectable by reverse transcription-PCR/nested PCR including, but not limited to, PDX-1, PAX-4, PAX-6, NRx2.2 and NRx6.1, insulin I, insulin II, glucose transporter 2 (GLUT2), and glucagons. Other agents may be added to this culture system for increasing the concentration of insulin-producing cells; polyamines, (Sjoholm, et. al., 1994, Endocrinology 135:1559); hepatocyte growth factor (Beattie, et al. 1996, Diabetes 45:1223); and, betacellulin (Cho, et. al., 2008, Biochem. Biophys. Res. Commun. 366:129). Various extracellular matrix components such as fibronectin and laminin may also be added to increase yield or concentration of insulin-producing cells (Leite, et al., 2008, Cell Tissue Res. 327:529).

The ability of the insulin-producing cells derived from ESSC to function in vivo may be studied using animal models or in clinical trials. A known model involves administration of putative insulin-producing cells into mice that have been treated with streptozoticin, which destroys insulin-producing β cells. Recipient mice may be immune suppressed or immune deficient, such as nude mice, RAG knockout, or SCID mice. Production of human C-peptide may be used as a proxy of insulin production, alternatively glucose responsiveness may be studied. An example of in vivo assessment of stem cell-derived insulin-producing cells is provided in Davani, et al., 2007, Stem Cells 25:3215.

The invention further provides conditioned medium and methods of producing conditioned medium. A conditioned medium is or has been in contact with (e.g., incubated) which a particular cell or population of cells for a period of time, and then removed, and thus can be produced accordingly. While the cells are cultured in the medium, they secrete cellular factors into the medium, such as by way of example, insulin, but also secrete additional factors. Conditioned medium and methods of producing conditioned medium additionally include concentrated (concentrating), lyophilized (lyophilizing) and freeze-dried forms (freeze drying). Such medium can be separated from cells by withdrawal from a cell culture, such as by aspiration or dispensing the medium, in a container or vessel.

In various embodiments, storing, stored, preserving and preserved pluripotent stem cells and conditioned medium include freezing (frozen) or storing (stored) ESSC and conditioned medium, such as, for example, individual ESSC or their progeny, a population or plurality of ESSC or their progeny, a culture of ESSC or their progeny, co-cultures and mixed populations of ESSC or their progeny and other cell types and conditioned medium. ESSC, their progeny, and their conditioned medium can be preserved or frozen, for example, under a cryogenic condition, such as at −20° C. or less, e.g., −70° C. Preservation or storage under such conditions can include a membrane or cellular protectant, such as dimethylsulfoxide (DMSO).

Mammalian ESSC, a population or plurality or culture of ESSC, progeny of ESSC (e.g., any clonal progeny or any or all various developmental, maturation and differentiation stages) and conditioned medium of ESSC cells can be used for various applications, can be used in accordance with the methods of the invention including treatment and therapeutic methods. The invention therefore provides in vivo and ex vivo treatment and therapeutic methods that employ mammalian ESSC, populations and pluralities and cultures of ESSC, progeny of ESSC and conditioned medium of ESSC.

Mammalian ESSC, a population or plurality or culture of ESSC, progeny of ESSC (e.g., any clonal progeny or any or all various developmental, maturation and differentiation stages) and conditioned medium of ESSC cells can be can be administered to a subject, or used to implant or transplant as a cell-based or medium based therapy, or to provide factors, such as secreted insulin to provide a benefit to a subject (e.g., by differentiating into cells in the subject, or stimulate, increase, induce, promote enhance or augment activity or function of endogenous insulin-producing cells).

### Therapy

The invention contemplates use of the cells of the invention in both in vitro and in vivo settings. Thus, the invention provides for use of the cells of the invention for research purposes and for therapeutic or medical/veterinary purposes. In research settings, an enormous number of practical applications exist for the technology. One example of such applications is use of the cells of the invention in an ex vivo diabetes model in a lab, thus avoiding use of ill patients to optimize a treatment method.

In accordance with the invention, methods of providing a cellular therapy and methods of treating a subject having a disease or disorder that would benefit from a cellular therapy are provided. In one embodiment, a method includes administering at least one insulin-producing cell derived from an ESSC to a subject in an amount sufficient to provide a benefit to the subject. In various embodiments, the subject having a disease or disorder has diabetes, such as diabetes type I, diabetes II or gestational diabetes. ESSC, the progeny of ESSC, or conditioned medium of ESSC or their progeny can be administered or delivered to a subject by any route suitable for the treatment method or protocol. Specific non-limiting examples of administration and delivery routes include parenteral, e.g., intravenous, intramuscular, intrathecal (intra-spinal), intraarterial, intradermal, subcutaneous, intra-pleural, transdermal (topical), transmucosal, intra-cranial, intra-ocular, mucosal, implantation and transplantation.

In some embodiments, the ESSC or their progeny can be autologous with respect to the subject; that is, the ESSC used in the method (or to produce the conditioned medium) were obtained or derived from a cell from the subject that is treated according to the method. In other embodiments, the ESSC, the progeny of ESSC or conditioned medium of ESSC or their progeny can be allogeneic with respect to the subject; that is, the ESSC used in the method (or to produce the conditioned medium) were obtained or derived from a cell from a subject that is different from the subject that is treated according to the method.

The methods of the invention also include administering ESSC, progeny of ESSC, or conditioned medium of ESSC prior to, concurrently with, or following administration of additional pharmaceutical agents or biologics. Pharmaceutical agents or biologics may activate or stimulate ESSC or their progeny. Non-limiting examples of such agents include, for example: erythropoietin, prolactin, human chorionic gonadotropin, gastrin, EGF, FGF, and VEGF.

The methods of the invention also include methods that provide a detectable or measurable improvement in a condition of a given subject, such as alleviating or ameliorating one or more signs or symptoms of a disease or disorder, such as, for example, diabetes.

In methods of treatment, a method may be practiced one or more times (e.g., 1-10, 1-5 or 1-3 times) per day, week, month, or year. The skilled artisan will know when it is appropriate to delay or discontinue administration. Frequency of administration is guided by clinical need or surrogate markers. An exemplary non-limiting dosage schedule is every second day for a total of 4 injections, 1-7 times per week, for 1, 2, 3, 4, 5, 6, 7, 8, 9, 10, 15, or more weeks, and any numerical value or range or value within such ranges.

Of course, as is typical for any treatment or therapy, different subjects will exhibit different responses to treatment and some may not respond or respond less than desired to a particular treatment protocol, regimen or process. Amounts effective or sufficient will therefore depend at least in part upon the disorder treated (e.g., the type or severity of the disease, disorder, illness, or pathology), the therapeutic effect desired, as well as the individual subject (e.g., the bioavailability within the subject, gender, age, etc.) and the subject's response to the treatment based upon genetic and epigenetic variability (e.g., pharmacogenomics).

The present invention also pertains to kits useful in the methods of the invention. Such kits comprise various combinations of components useful in any of the methods described elsewhere herein, including for example, hybridization probes or primers (e.g., labeled probes or primers), antibodies, reagents for detection of labeled molecules, materials for the amplification of nucleic acids, medium, media supplements, components for deriving an insulin-producing cell derived from an ESSC, an ESSC cell, and instructional material. For example, in one embodiment, the kit comprises components useful for deriving an insulin-producing cell from a stem cell.

The invention further provides kits, including ESSC, populations or a plurality of ESSC, cultures of ESSC, co-cultures and mixed populations of ESSC, progeny differentiated ESSC of any developmental, maturation or differentiation stage, as well as conditioned medium produced by contact with ESSC or their progeny, packaged into suitable packaging material. In various non-limiting embodiments, a kit includes an insulin-producing cell derived from an ESSC. In various aspects, a kit includes instructions for using the kit components e.g., instructions for performing a method of the invention, such as culturing, expanding (increasing cell numbers), proliferating, differentiating, maintaining, or preserving ESSC or their progeny, or a cell based treatment or therapy. In various aspects, a kit includes an article of manufacture, for example, an article of manufacture for culturing, expanding (increasing cell numbers), proliferating, differentiating, maintaining, or preserving ESSC or their progeny, such as a tissue culture dish or plate (e.g., a single or multi-well dish or plate such as an 8, 16, 32, 64, 96, 384 and 1536 multi-well plate or dish), tube, flask, bag, syringe, bottle or jar. In additional various aspects, a kit includes an article of manufacture, for example, an article of manufacture for administering, introducing, transplanting, or implanting pluripotent stem cells into a subject locally, regionally or systemically.

A label or packaging insert can include appropriate written instructions, for example, practicing a method of the invention. Thus, in additional embodiments, a kit includes a label or packaging insert including instructions for practicing a method of the invention in solution, in vitro, in vivo, or ex vivo. Instructions can therefore include instructions for practicing any of the methods of the invention described herein. Instructions may further include indications of a satisfactory clinical endpoint or any adverse symptoms or complications that may occur, storage information, expiration date, or any information required by regulatory agencies such as the Food and Drug Administration for use in a human subject.

The instructions may be on “printed matter,” e.g., on paper or cardboard within the kit, on a label affixed to the kit or packaging material, or attached to a tissue culture dish, tube, flask, roller bottle, plate (e.g., a single multi-well plate or dish such as an 8, 16, 32, 64, 96, 384 and 1536 multi-well plate or dish) or vial containing a component (e.g., pluripotent stem cells) of the kit. Instructions may comprise voice or video tape and additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

The kits of the invention can additionally include growth medium, buffering agent, a preservative, or a cell stabilizing agent. Each component of the kit can be enclosed within an individual container or in a mixture and all of the various containers can be within single or multiple packages.

ESSC or their progeny, as well as conditioned medium produced by contact with ESSC or their progeny, can be packaged in dosage unit form for administration and uniformity of dosage. “Dosage unit form” as used herein refers to physically discrete units suited as unitary dosages; each unit contains a quantity of the composition in association with a desired effect. The unit dosage forms will depend on a variety of factors including, but not necessarily limited to, the particular composition employed, the effect to be achieved, and the pharmacodynamics and pharmacogenomics of the subject to be treated.

ESSC or their progeny, as well as conditioned medium produced by contact with ESSC or their progeny, can be included in or employ pharmaceutical formulations. Pharmaceutical formulations include “pharmaceutically acceptable” and “physiologically acceptable” carriers, diluents or excipients. The terms “pharmaceutically acceptable” and “physiologically acceptable” mean that the formulation is compatible with pharmaceutical administration. Such pharmaceutical formulations are useful for, among other things, administration or delivery to, implantation or transplant into, a subject in vivo or ex vivo.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical formulations can be made to be compatible with a particular local, regional or systemic administration or delivery route. Thus, pharmaceutical formulations include carriers, diluents, or excipients suitable for administration by particular routes. Specific non-limiting examples of routes of administration for compositions of the invention are parenteral, e.g., intravenous, intramuscular, intrathecal (intra-spinal), intraarterial, intradermal, subcutaneous, intra-pleural, transdermal (topical), transmucosal, intra-cranial, intra-ocular, mucosal administration, and any other formulation suitable for the treatment method or administration protocol.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Supplementary compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents.

Preservatives can be used to inhibit microbial growth or increase stability of ingredients thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

Pharmaceutical formulations and delivery systems appropriate for the compositions and methods of the invention are known in the art (see, e.g., Remington: The Science and Practice of Pharmacy (2003) 20.sup.th ed., Mack Publishing Co., Easton, Pa.; Remington's Pharmaceutical Sciences (1990) 18.sup.th ed., Mack Publishing Co., Easton, Pa.; The Merck Index (1996) 12.sup.th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11th ed., Lippincott Williams & Wilkins, Baltimore, Md.; and Poznansky et al., Drug Delivery Systems (1980), R. L. Juliano, ed., Oxford, N.Y., pp. 253-315).

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Derivation of Insulin-Producing Cells from Human Endometrial Stromal Stem Cells and their Use in the Treatment of Murine Diabetes

As described herein, human ESSCs have the potential to be reprogrammed into insulin-producing cells. Moreover, with a view to clinical application, this protocol avoids gene transfection or other genetic manipulation. This offers, for example, a potential therapeutic tool to treat diabetic women, providing each woman with her own immunologically matched stem cells, as well as treatment of men after matching with the vast numbers of these cells discarded from hysterectomy specimens.

Pancreatic islet cell transplantation is an effective approach to treat type 1 diabetes, however the shortage of cadaveric donors and limitations due to rejection require alternative solutions. Multipotent cells derived from the uterine endometrium have the ability to differentiate into mesodermal and ectodermal cellular lineages, suggesting the existence of mesenchymal stem cells in this tissue. Human endometrial stromal stem cells (ESSC) were differentiated into insulin secreting cells using a simple and nontransfection protocol. An in vitro protocol was developed and evaluated by assessing the expression of pan β-cell markers, followed by confirmation of insulin secretion. PAX4, PDX1, GLUT2, and insulin were all increased in differentiated cells compared to controls. Differentiated cells secreted insulin in a glucose responsive manner. In a murine model, differentiated cells were injected into the kidney capsules of diabetic mice and human insulin identified in serum. Within 5 weeks blood glucose levels were stabilized in animals transplanted with differentiated cells, however those treated with undifferentiated cells developed progressive hyperglycemia. Mice transplanted with control cells lost weight and developed cataracts while those receiving insulin-producing cells did not. Endometrium provides an easily accessible, renewable, and immunologically identical source of stem cells with potential therapeutic applications in diabetes.

A three-step protocol was developed to effectively induce the differentiation of ESSCs into insulin-producing cells, using defined media only, and avoiding transfection. This method was chosen with a view to a potential therapeutic application. To test the applicability of these cells for medical therapy, their functionality was confirmed in both in vitro and in vivo models.

Several factors appear to influence the efficiency of the protocol in ESSC differentiation. First, the aggregation and clustering on ECM in the second step of the protocol seemed to be crucial for the complete differentiation in culture. Cells cultured without ECM gel did not form islet-like clusters. Indeed, it has been previously shown that ECM gel coated-dishes support the migration of pancreatic progenitor cells, formation of 3-D cystic structures and protrusion of islet buds (Kaido et al., 2010, Chinese Med. J. 121:811-818; Chen et al., 2004, World J. Gastroenterol. 10:3016-3020). Second, the treatment with indolactam V was most effective when introduced at the same stage as reported by Chen et al., when the gut endodermal associated gene, NGN3, was first expressed, but before peak PDX1 expression (Kaido et al., 2010, J. Cell Physiol. 224:101-111). Finally, addition of Exendin-4 and Activin A in the third step yielded higher expression of β cell pan-markers. Exendin 4 is a potent GLP-1 agonist that stimulates both β cell replication and neogenesis from ductal progenitor cells (Oh et al., 2004, Cell 131:861-872). Interestingly, the absence of Activin A from step 3 results in glucagon producing cells (a cells), hence supporting the already established paradigm that addition of Activin A diverts the differentiation towards a Pax-4 expressing pancreatic lineage (Collombat et al., 2009, Cell 138:449-462, St-Onge, et al., 1997, Nature 387:406-409; Sosa-Pineda, et al., 1997, Nature 386:399-402).

Previous reports have demonstrated the ability of adult stem cells to stabilize and normalize the blood glucose levels in STZ treated rodents (Ramiya et al., 2000, Nat. Med. 6:278-282). These autologous treatments, however, require a complicated procedure in order to harvest these cells. Optimally, to facilitate wide adoption, stem cell acquisition will require an easily accessible and viable pool of stem cells. The cells should be obtained by a simple procedure that will not compromise health. The method described herein provides an easily accessible source of stem cells obtainable through a simple, routine office biopsy procedure. Uterine endometrium is unique in that it is completely regenerated in each menstrual cycle or in response to estrogen treatment even in menopausal women; the virtually inexhaustible supply is a clear advantage to the use of these cells. In addition, cells harvested from hysterectomy specimens can be stored and matched for use in men or women. The results described herein further support the regenerative capability of endometrial stem cells and demonstrate the use of endometrium as a potential source of progenitor cells for therapeutic application.

The materials and methods employed in these experiments are now described.

### Materials and Methods

### Cell Culture and Flow Cytometry Analysis

All procedures were conducted according to institutionally approved human investigative committee protocols. Human endometrial tissue was obtained from seven patients (n=7). Tissue was processed using. Hank's balanced salt solution (Gibco, Carlsbad, Calif.) containing 4-(2-hydroxyethyl)-1-piperazineethanesulfonic acid (25 mmol/l), collagenase B (1 mg/ml; Roche Diagnostics, Indianapolis, Ind.), and DNase I (0.1 mg/ml; Sigma-Aldrich, St Louis, Mo.) for 60-90 minutes at 37° C. with agitation. Cells were plated in media containing DMEM/F12 (Gibco-Invitrogen, Carlsbad, Calif.) with 10% fetal bovine serum (FBS) (Invitrogen, Carlsbad, Calif.), 1% penicillin-streptomycin (Gibco), 1% amphotericin B (American Bio, Natick, Mass.) and incubated at 37° C. in 5% CO₂. Endometrial cells were expanded from passage 2-5 to allow 80% confluence. Before differentiation cultures were depleted from epithelial and hematopoietic cells.

Cultures were treated with antibodies as previously described (Wolff et al., 2011, J. Cell. Mol. Med. 15:747-755) and analyzed by flow cytometry, using FACS Vantage SE (BD Biosciences, San Jose, Calif.). The results were analyzed using the FlowJo software.

Differentiation into Insulin-Producing Cells

Briefly, in step one, the cell monolayer was treated for 24 hours with high glucose Dulbecco's modified Eagle medium (DMEM) 25 mmol/l, 10% FBS and 10⁻⁶ mol/l retinoic acid (Sigma-Aldrich), followed by 2 days treatment in media containing high glucose DMEM and 10% FBS for 2 days. In step two, cells were first detached with 0.05% trypsin and seeded in plates precoated with ECM gel from Engelbreth-Holm-Swarm murine sarcoma (Sigma-Aldrich). Cells were cultured in medium that consisted of low glucose DMEM 5.56 mmol/l, 10% FBS, 10 mmol/l nicotinamide (Sigma-Aldrich), 20 ng/ml epidermal growth factor (PeproTech, Rocky Hill, N.J.), 50 ng/ml of FGF-10 (R&D, Minneapolis, Minn.), and 300 nmol/l of (−)-indolactam V (LC laboratories, Woburn, Mass.) for 9 days. In step three cells were cultured in L-DMEM, 10% FBS, 10 nmol/l exendin-4 (Sigma-Aldrich), and 50 ng/ml Activin A (PeproTech) for 7 days. All the media above described were supplemented with 1% penicillin/streptomycin and 1% amphotericin B. Control cultures were grown in media containing DMEM/F12, 1% penicillin/streptomycin, 1% amphotericin B, and 10% FBS.

### RT-PCR

The cells were detached after a 2-hour digestion with dispase (BD Biosciences, San Jose, Calif.) and mRNA was extracted using the RNeasy kit (Qiagen, Valencia, Calif.). Subsequently, mRNA was reverse transcribed using Superscript and gene expression analysis was performed with RT-PCR.

The following genes were amplified using qRT-PCR: Insulin (INS), Glucagon (GCG), HNF6a, PDX1, NGN3, PAX4, GLUT2 and β-actin as an internal control. All PCR experiments were performed using Taq Polymerase (SYBR-Green) and a Bio-Rad qPCR detection system (Bio-Rad Laboratories, Hercules, Calif.).

Products were also analyzed by 1.5% agarose gel electrophoresis. The sequences of the primers, PCR products, number of cycles and annealing temperature are listed in Table 1.

TABLE 1
Primers for RT-PCR

| | | Annealing | Product's | |
|---|---|---|---|---|
| Gene | Primer's sequence | temperature | size in bp | |
| Insulin FW | GCCCTCCAGGACAGGCTGC | 59.3 | 259 | SEQ ID NO: 1 |
| Insulin RV | CTCCACCTGCCCCACCTGC | 59.2 | 259 | SEQ ID NO: 2 |
| Hnf6a FW | GCATGTCCGCGCTCCGCTTA | 60.1 | 270 | SEQ ID NO: 3 |
| Hnf6a RV | TGGAGCTGCCCTCGTCCTGC | 60.8 | 270 | SEQ ID NO: 4 |
| PDX-1 FW | ACCACCACCTCCCGGCTCAG | 60.1 | 203 | SEQ ID NO: 5 |
| PDX-1 RV | GCGCGTCCGCTTGTTCTCCT | 60.0 | 203 | SEQ ID NO: 6 |
| Ngn3 FW | GCGACCAGAAGCCCGCTGAG | 60.1 | 210 | SEQ ID NO: 7 |
| Ngn3 RV | GTTCCCCCGTGTGCGAGTGG | 59.9 | 210 | SEQ ID NO: 8 |
| Glut2 FW | CAGCTGGCCATCGTCACGGG | 60.1 | 111 | SEQ ID NO: 9 |
| Glut2 RV | GGCTCGCACACCAGACAGGC | 60.3 | 111 | SEQ ID NO: 10 |
| β-actin FW | ACAGAGCCTCGCCTTTGCCG | 59.7 | 190 | SEQ ID NO: 11 |
| β-actin RV | CACCATCACGCCCTGGTGCC | 60.3 | 190 | SEQ ID NO: 12 |
| PAX-4 FW | GGGGTACCCACCCAGGGACC | 59.8 | 240 | SEQ ID NO: 13 |
| PAX-4 RV | CCTGGGAAGCACCTGGCAGC | 59.9 | 240 | SEQ ID NO: 14 |

Abbreviations: bp, base pair; RT-PCR, reverse-transcripitase PCR.

### Immunocytochemistry

Samples were fixed with 4% paraformaldehyde. Subsequently, cells were blocked in 5% goat serum (Sigma-Aldrich) and 1% bovine serum albumin in phosphate-buffered saline to eliminate endogenous globulin signal and incubated with mouse monoclonal antibody against human insulin (1:100 dilution) (Abcam, Cambridge, Mass.) at 4° C. overnight, washed multiple times with phosphate-buffered saline and finally incubated with a secondary goat FITC conjugated anti-mouse antibody (Abeam) for 1.5 hour in room temperature. Samples were visualized with a contrast microscope (Zeiss, Thornwood, N.Y.) using a wavelength appropriate to the fluorophore dyes.

### Insulin Detection Assay

Insulin release upon treatment with different glucose concentrations (5.5 mmol/L and 28 mmol/L) was measured using ELISA (Millipore, Billerica, Mass.). Plated cells on ECM gel were washed twice and incubated in serum free media containing 5.5 mmol/L glucose for 4 hours at 37° C. The media was collected and the cells were washed and incubated for 4 more hours at 37° C. in serum-free media containing 28 mmol/L. All the collected media were stored at −80° C. until the ELISA measurements for human insulin were performed (Seeberger et al., 2006, Lab. Invest. 86:141-153; Chen et al., 2009, Nat. Chem. Biol. 5:258-265).

### Animal Model

All animal procedures were conducted according to Yale Institutional Animal Care and Use Committee approved protocols and guidelines. 8-10 week-old female severe combined immunodeficiency/nonobese diabetic mice (Jackson Laboratory, Bar Harbor, Me.) were treated with 180 mg/kg of STZ (Calbiochem, San Diego, Calif.) administered as a single intraperitoneal injection. Blood glucose Level was determined using a standard blood glucose meter (Accu-check). Hyperglycemia developed 5-7 days following the STZ injection. Mice were considered diabetic if two random blood glucose measurements were >220 mgr/ml.

Under anesthesia, diabetic mice received a renal subcapsular transplant of 10⁷ differentiated cells (n=8) or 10⁷ of nondifferentiated cells (n=8), 7-10 days after STZ injection. Nontreated severe combined immunodeficiency mice (n=6) and diabetic mice treated with STZ only (n=5) were used as controls. Fasting blood glucose levels were measured weekly after surgery for 6 weeks.

### Immunohistochemistry

Mice were euthanized, left kidneys were removed, fixed in 4% paraformaldehyde and were paraffin embedded. Kidneys were transversally sectioned (5 μm thickness) and blocked by using biotin-avidin blocking kit (Vector Labs, Burlingame, Calif.). The staining for insulin was done by using M.O.M. kit (Vector Labs) with mouse monoclonal antibody against human insulin (1:500) (Abcam) and according to Vector Labs published protocol. Finally, the slides were mounted with Vectashield (Vector Labratories) and 4′,6-diamidino-2-phenylindole and visualized by using NLO Confocal Microscope (Zeiss).

### Human Insulin Radioimmunoassay

Human Insulin Production was assessed in mice blood serum by Radioimmuno Assay (RIA). For that purpose, an ultrasensitive human insulin RIA kit without cross reactivity between mouse and human insulin was used (Millipore, Billerica, Mass.). Blood samples from the retro-orbital plexus were collected from each non-fasting mouse and a pool of plasma from each experimental group was assessed.

### Statistics

Statistical analysis was performed using analysis of variance, and P<0.05 was considered significant. For RT-PCR, we performed a standard curve in order to assess the absolute expression of each of the samples for each of the genes. Each amplification product's mass (in nanograms) was assessed from the linear curve, followed by calculations of ratios of inspected genes to the reference gene, β-actin.

The results of the experiments are now described.

### Characterization of Endometrial Stem Cell Population

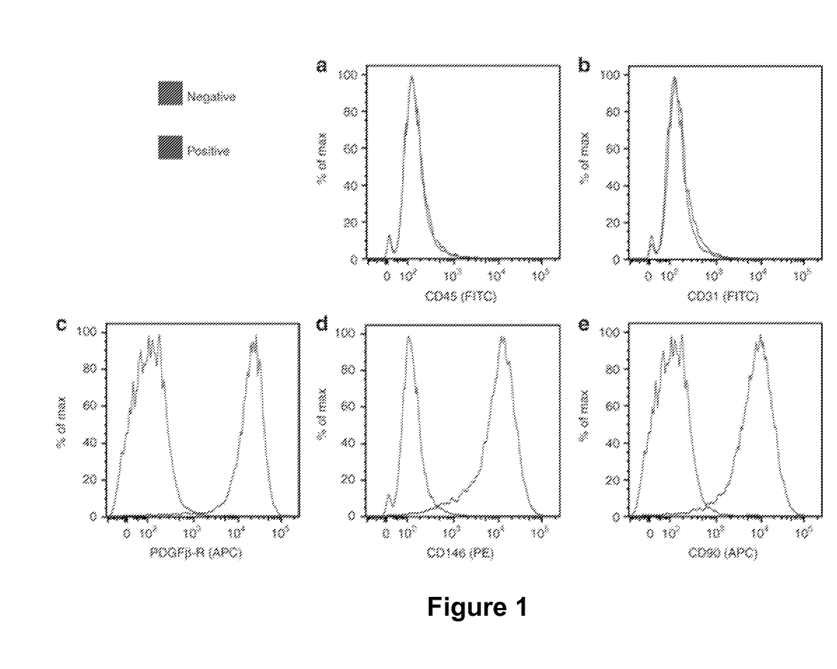

Endometrial stromal cells were sorted by fluorescence-activated cell sorting. The enrichment in PDGFβ-R⁺/CD146⁺/CD90⁺/CD45⁻/CD31⁻ human ESSC cells was achieved as we have previously described (Wolff et al., 2011, J. Cell Mol. Med. 15:747-755) (FIGS. 1A-1E). This protocol to isolate endometrial stem cells has been previously used in the derivation of osteogenic and chondrogenic lineages as well as in a previous report describing the generation of dopaminergic neurons (Wolff et al., 2011, J. Cell Mol. Med. 15:747-755). 2-3×10⁷ cells were obtained from fresh endometrial tissue and plated in a monolayer and passaged twice, in order to deplete the culture from CD45⁺/CD31⁺ and epithelial cells. Successful differentiation of human ESSCs was conducted via three steps protocol, as described elsewhere herein.

In Vitro Differentiation of Insulin-Producing Cells from hESSC

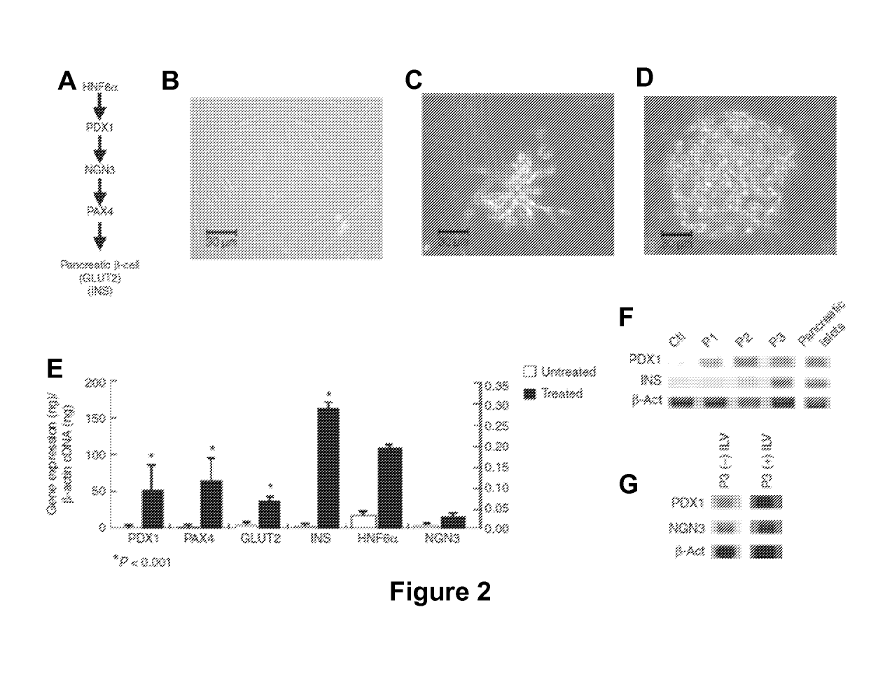

In order to derive pancreatic β-like cells from endometrial stem cells, three established differentiation protocols were compared. After each stage of differentiation, the expression of genes that are associated with pancreatic cell differentiation was analyzed (FIG. 2A).

No change in cellular morphology was observed by the end of step one (FIG. 2B). However, once the cells were plated on extracellular matrix (ECM) and exposed to step two media, islet-like clusters appeared (FIG. 2C). By the end of step three, the islet-like clusters from step two increased in size and number as well as developed typical islet morphology (FIG. 2D).

Reverse-transcriptase PCR (RT-PCR) results demonstrated that cells completing step three had increased expression of pancreatic lineage markers compared with the undifferentiated cells (control) (FIG. 2E). Levels of PDX1, PAX4, GLUT2 and Insulin (INS) mRNA all increased significantly in the treated cells (P<0.001) and demonstrated the effectiveness of the differentiation protocol. The expression of PAX4 confirmed that the protocol favored the β-cell lineage fate.

Although not wishing to be bound by any particular theory, the relatively minor change by the end of step three in the expression of HNF6a and NGN3, with respect to the control (FIG. 2E, right y-axis), suggested that most of the cells in the culture were mature, as these genes are developmentally associated only with early stages of pancreatic differentiation. To examine the progression of maturation in each culture media (step 1-3), the expression of insulin (associated with mature β cells) and PDX1 were determined at each step. Unlike the expression of insulin, which increased in step three, PDX1 expression increased throughout the treatment (FIG. 2F).

In order to increase the yield of cells that expressed pancreatic β-cells pan-markers, the small molecule indolactam V was used, which has been shown to enhance β-cell differentiation, and result in a significantly higher number of PDX-1 expressing cells (Chen et al., 2009, Nat. Chem. Biol. 5:258-265). The incorporation of indolactam V into step two of the protocol, when PDX1 expression is first expressed, indeed resulted in higher expression levels, followed by a similar trend in the expression of NGN3 (FIG. 2G).

### Insulin Release in Response to Glucose Stimulation

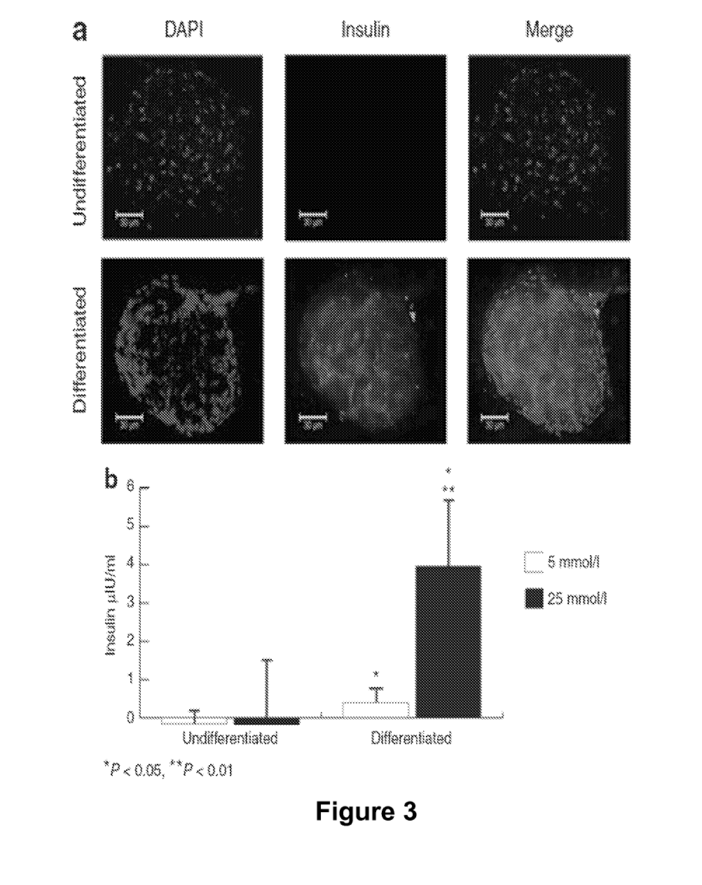

Human ESSCs in culture were subjected to immunocytochemistry, using antibodies specific against human insulin. All samples were washed three times with phosphate-buffered saline to avoid insulin that may have been present in the media. Insulin was not detected in undifferentiated cultures whereas a strong signal was found in the clusters of treated cells (FIG. 3A).

To determine whether insulin production was glucose-dependent, differentiated cells were treated with two concentrations of glucose. Enzyme-linked immunosorbent assay was performed to measure secretion of insulin in response to different levels of glucose in the cell culture media. While undifferentiated endometrial cultures did not release insulin in the presence or absence of a glucose challenge (FIG. 3B), differentiated cells responded with secretion of insulin. Furthermore, the differentiated cells responded by secreting insulin in a glucose dependent manner. Insulin production was increased by approximately tenfold in response to an increase in exposure to glucose from 5 to 25 mmol/l (P<0.05). Nevertheless, the total amount of secreted insulin was in the μIU/ml range, which is consistent with the explanation that only a small number of human ESSCs were completely differentiated into insulin-producing cells.

### Transplantation of Insulin Secreting Cells to Diabetic Mice

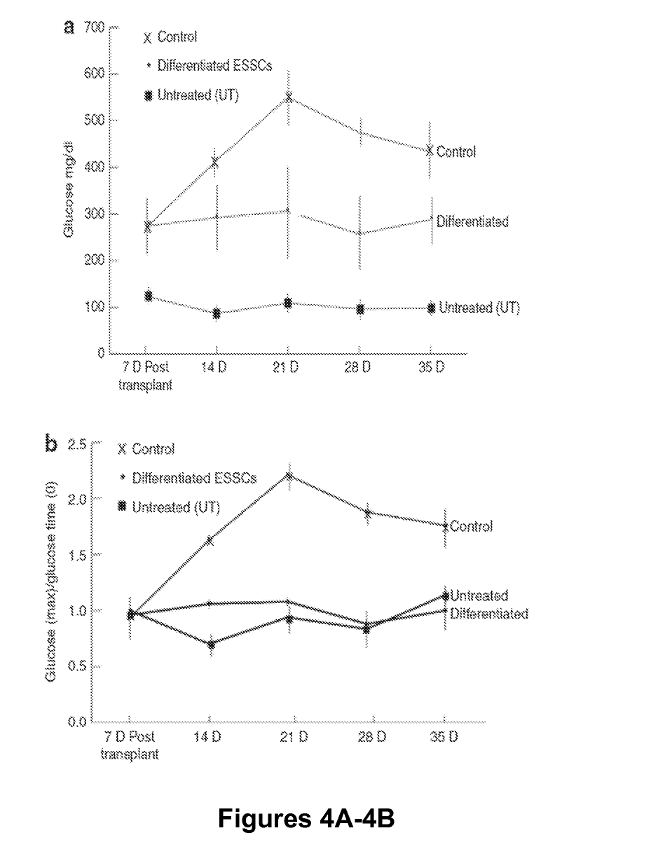

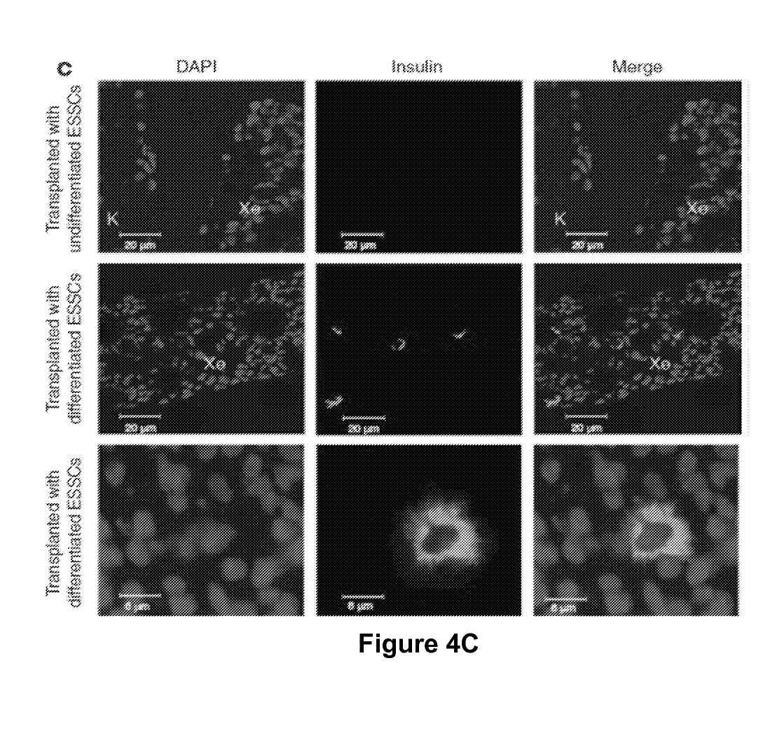

To assess the potential use of these cells for therapeutic purposes in patients with type 1 diabetes, their effectiveness in diabetic severe combined immunodeficiency mice was tested (FIGS. 4A-4B). Animals were first injected with streptozotocin (STZ) 7 days before transplant with differentiated human ESSC. Mice with blood glucose measurement levels above 220 mg/dl in response to STZ treatment were chosen as subjects for the experiment. The first group of animals (n=8) was injected with ESSCs that had been treated with the differentiation protocol described above, while the second group (control) (n=8) was transplanted with undifferentiated cells. As a negative control, a third group of animals (n=6) was treated with STZ to induce diabetes, but did not undergo cell transplant surgery. Lastly, a fourth group of severe combined immunodeficiency mice (n=5) were untreated with STZ and did not undergo surgery (UT).

In the untreated group (UT) blood glucose levels remained low as expected (FIG. 4A). In the control group that received transplants of undifferentiated cells, glucose levels increased significantly to a peak by the end of the fourth week after the transplant. Although not wishing to be bound by any particular theory, the elevation in the glucose levels of the control group might be exacerbated partially in response to stress caused by the surgery. The group that was treated with differentiated ESSCs showed a stabilization of glucose at the level induced by STZ before the time of transplant. The increase after surgery seen in the controls was prevented. A further decrease in glucose levels was not observed. To illustrate the stabilization of blood glucose, FIG. 4B shows the mean blood glucose levels after transplant as a proportion of the glucose level at the time of transplant (Glu T_{X}/Glu T₀). The control group transplanted with undifferentiated cells had rising glucose levels. In contrast, the glucose level of the group that was treated with differentiated cells was stable and equivalent to that of the untreated group (UT).

Finally, kidney capsules from each of the two transplanted groups were subjected to immunofluorescent staining using antibodies that recognize only human insulin to confirm insulin production from the engrafted cells (FIG. 4C). While no insulin signal was observed in the kidney capsules of the control group that were implanted with undifferentiated cells (upper panel), in the kidneys that were engrafted with differentiated ESSCs, insulin production from single cells within the xenograft were observed (middle and lower panels).

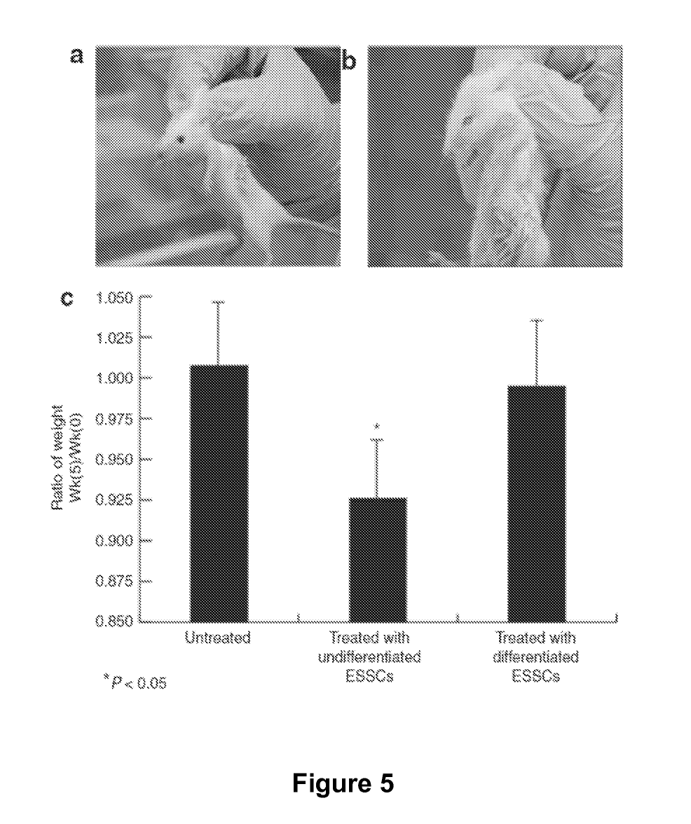

Mice from the group that were transplanted with differentiated cells displayed no gross pathological symptoms (FIG. 5A) and were comparable to the untreated group (UT). In contrast, diabetic mice that were either not transplanted or transplanted with undifferentiated ESSCs showed a large number of complications in the initial observation period (FIG. 5B). Complications seen in all diabetic mice that did not receive stem cell transplant included cataracts, obvious signs of dehydration, loss of skin resiliency and prolonged recovery time after pinching, loss of fur sheen and passive, sedate behavior. In addition, weight measurements were conducted twice during the monthly follow up and averaged (FIG. 5B). The group that was treated with the undifferentiated cells suffered significant weight loss (P<0.05), unlike the group that was treated with the differentiated cells that showed no significant difference compared to the untreated group (UT).

To assess the levels of human insulin in the animal's serum, blood samples were drawn and subjected to radioimmunoassay. Results showed mean human insulin level of 11.9 μIU/ml in the blood of the mice that were treated with differentiated cells whereas no human insulin was detected in the other three groups. The amount of insulin measured in the group that was transplanted with treated cells was sufficient to prevent mice from developing diabetes mellitus-related complications after a 4-week follow up period.

### Example 2

### Clinical Grade Cell Population

The cells of the invention can be generated using a series of systems and standard operating procedures that allow for creation of a purified, clinical grade cell population. In one embodiment of the invention, human endometrial tissue is collected. Each subject's collection schedule is coordinated with the study coordinator who arranges for courier pick up of the sample from the clinic to the laboratory.

The sample is transferred at the clinic into an appropriate buffer. At the laboratory (General BioTechnology, LLC), the collection tube containing the sample is transferred to a 50 ml conical tube and filled to the top with GMP manufactured Phosphate Buffered Saline (PBS) and centrifuged at 500×g for 10 minutes. All supernatant is removed and the tube is filled to the top with PBS and centrifuged again at 500×g for 10 minutes. Once the supernatant is removed, the pellet is re-suspended in 15 mL DME/F-12 with 10% FBS. The cells are plated in a T75 flask and placed in the 37° C. incubator.

In one embodiment, the cells are cultured for a period of 1-30 days, more specifically, the cells may be cultured for 16 days in DMEF-12 with approximately 10% FBS (the culture is 70% confluent and passage 0). Cells are detached using TrypZean and 3 vials of 1M cells per vial are frozen. For cell expansion, one vial of the passage 0 cells is thawed and plated into a T225 tissue culture treated flask. The cells are cultured for 3-4 days between each splitting, and one vial is frozen at each P1, P2, and P3. For cryopreservation, cells are collected and equilibrated in a 10% GMP manufactured dimethyl sulfoxide (DMSO) solution, added step wise over 10 minutes. Cells are then packaged into cryovials and cooled at a controlled rate of −1° C./minute to −80° C. and then placed into vapor phase LN2 for storage. One passage 3 vial is thawed and cultured until passage 6, splitting every 3-4 days between each passage. At passage 6, vials (24 total) are cryopreserved and one T225 flask (1 M cells or ˜4500 cells per cm²) is plated for passage 7 in antibiotic free media. Once 70% confluent, four passage 7 vials are frozen down, and passage 6 and 7 vials are stored for the next expansion for the mice trials (MCB). The four passage 7 vials are used for each of the four days the mice will be transplanted. The passage 7 vials are thawed over 4 consecutive days, thawing one vial each day. Once plated, each culture is split every 3 days through passage 9. Once passage 9 is on the third day of culture, cells are harvested and split among 3 vials. One vial contains 5M cells, one vial contains 1 M cells and one vial contains 0.15M cells, all re-suspended in 125 μl Isolyte S injectable saline solution (the transplant vehicle).

Cells are then couriered to a facility for murine injection over 4 consecutive days (each day they receive the same 3 doses). The second round of mouse trials is performed in an identical fashion, thawing 1×1 M cell vial of passage 6 cells, culturing 3 days and freezing 4 vials of passage 7 cells. Once the mouse trial is ready, each of the 4×passage 7 vials are thawed over 4 consecutive days, cultured 3 days between each splitting and harvested at passage 9. Cell aliquots from each donor batch meet the following release criteria: (i) negative for bacterial and mycoplasma contamination; (ii) endotoxin levels <1.65 EU/ml; (iii) morphology consistent with adherent, fibroblastic-like shape; (iv) Cell viability >70% by 7-AAD staining. Mycoplasma, endotoxin and sterility are tested using validated contract laboratories. Cells are observed directly for morphology over the course of the expansion. Remaining passage 9 cells are centrifuged and re-suspended in 1×PBS, and counted on a hemocytometer. For clinical development, the use of a master cell bank is contemplated. The Master Cell Bank (MCB) may be generated from Passage 3 cells that are frozen down in CellSeal vials, containing 1 million cells per vial, with approximately 200 vials according to MS-CM-010. The MCB is stored at −180° C. in liquid nitrogen temperature monitored containers. A flow cytometry test is done on a sample of the MCB cells (10% of product).

For production of cells, reagent qualification may be necessary. The qualification process begins with the vender of the reagent. The vender is qualified through our standard operating procedure. A corresponding form is completed and approval gained before a vender can be used. The Criteria identified as important in qualifying a supplier include quality of product, services offered, competitive pricing, communication, availability, how complaints are handled and the overall fit to our systems. This list is not all inclusive. Quality Systems reviews each qualification form and will approve based on the criteria stated above. Once the vender is approved, they are added to the Supplies and Services List. Associates ordering supplies including reagents use the list. Only approved venders on the list are used by associates ordering supplies involving reagents. Once the reagent arrives, it is logged on the Supplies Receipt, Inspection and Inventory Log. The form instructs the associate to complete certain information for the incoming reagent. These fields are date received, initials of receiver, name of the item, manufacturer, lot number, expiration date, package passed visual inspection, product passed visual inspection, date available for use and quantity. The COA is examined for reagents and placed in the applicable COA binder under that reagent name. These binders are retained per the record retention procedure. Once this is completed the reagent is released from quarantine and placed in the applicable area. If the reagent needs refrigerated or is to remain frozen, it is placed in the applicable storage environment. FDA or other national regulatory body-approved reagents are used if available. In one embodiment, an excipient used in the cryopreservation of the cells is Dimethyl Sulfoxide (DMSO). Each dose of cell is cryopreserved using 10% DMSO, or 2 mL of DMSO in a total volume of 10 mL of final product. Infusion of this amount of DMSO is well within the safety parameters for a 30 kg child; Pediatric Stem Cell Transplant SOP states that the maximum dose of DMSO is 15 mg/kg/dose.

During the process of manufacturing, it is ideal for the production to occur in a class 10,000 clean production suite. Each technician properly gowns when entering in the GMP room. Before entry into the clean lab area, the technician obtains a bunny suit in the ante room. After the hood of the bunny suit is placed on, a mouth covering is put on, making sure that all hair is fully covered under the hood and mouth covering. The technician puts on a pair of sterile powder free gloves, and entera the clean lab space with the sample. Environmental monitoring is performed in the Class 10,000 clean room. The clean room uses Biological Safety Cabinets (BSC) which maintains a Class 5 environment. BSC are certified annually by an outside qualified vender. Settling plates are performed every time the BSC is in use for processing and evaluated for acceptable criteria based on USP. One settling plate is placed in the BSC during processing for a minimum of 30 minutes. Once per package, as a negative control, one covered settling plate is placed inside the BCS at the same time. After the settling plate is in the BSC, evaluate the plate for presence of bacterial colonies, Colony Forming Units (cfu), by allowing the plate to incubate for 48 hours at 37° C. Levels requiring alert are more than 1 colony per plate. Incubator temperature should be 36-38° C. TSA plates are used to evaluate the wide spectrum of possible bacteria present. Prepared plates are in their original wrapping at 2-8° C. and are warmed to room temperature prior to use. The product is validated from the time of manufacture to be stable at room temperature (25° C.) for 192 hours (8 days). Additionally the clean room is monitored for room temperature and particle counts. Acceptable room temperature is between 15 and 30 degrees Celsius. A MetOne Aerocet 531 particle counter, or alternative, may used to evaluate the particles in the air. The particle counter is used to detect and count the number of particles found in the air of the clean room. It is used to confirm that the number of loose particles in the air is less than 10,000 0.5 micron particles per ft³. The particle counter is run on a weekly basis in the three major areas of the clean room space. It is run for 30 minutes each in the gowning area, on the counter inside the clean room space and inside the hood. A settle plate is placed each time the particle counter is in use, next to the counter for the 30 minutes it is being run. After each use of the clean room, the BSC is wiped down with 5.25% bleach then followed by a 70% isopropyl alcohol. Countertops inside the clean room space are wiped down with 70% isopropyl alcohol each day. Once a week all surfaces inside the clean room, including floor, are wiped down with enzymatic cleaner LpH using a dry disposable cloth. Yearly, all walls and ceiling are clean with a lint roller, and all soft walls are cleaned with 70% isopropyl alcohol. Before laboratory technicians are allowed into the clean room, a gowning competency must be passed. RODAC plates are utilized to assess the competency of the technician. The acceptable limits of CFU/plate are determined according to local regulations. In one example, the following limits are used: Finger tips 10, CFU/plate, Gown Zipper 5 CFU/plate, Gown Lower Sleeve Area 5 CFU/plate, Hood Corner 5 CFU/Plate, Floor Surface 10 CFU/plate.

In another example, cell isolation begins with the delivery of the sample to the processing lab. Washing Tube containing the menstrual blood sample is topped up to 50 ml with PBS in the Biological Safety Cabinet and cells are washed by centrifugation at 500 g for 10 minutes at room temperature, which produces a cell pellet at the bottom of the conical tube. Under sterile conditions supernatant is decanted and the cell pellet is gently dissociated by tapping until the pellet appeared liquid. The pellet is re-suspended in 50 ml of PBS and gently mixed so as to produce a uniform mixture of cells in PBS. The cells are washed again by centrifugation at 500 g for 10 minutes at room temperature. Under sterile conditions, the supernatant is decanted and the cell pellet is re-suspended in 15 mL complete DME/F-12 media (Hyclone) supplemented with 10% Fetal Bovine Serum (Atlas Biologicals specified to have Endotoxin level: <=100 EU/ml (levels routinely <=10 EU/ml) and hemoglobin level: <=30 mg/dl (levels routinely <=25 mg/dl). The serum lot used is sequestered and one lot is used for all experiments. Additionally, the media is supplemented with 1% penicillin/streptomycin and 0.1% amphotericin B. The sample is plated in a T75 flask and placed in a 37° C. incubator. Media is changed after 24 hours, and then every 2-3 days at the discretion of the laboratory staff. Once cells reach 70-80% confluent, they are frozen down for quarantine (minimum 2 vials) and a culture screen is completed. The expended media from the culture is sent for sterility and mycoplasma testing. For sterility testing, the media is placed in 2 SPS tubes (1 mL media per tube) and sent to the testing facility. Cells from the sample are aliquotted into 2 vials which are frozen in 2 CellSeal 2.0 ml cryovials containing approximately 1 million cells per vial. Freezing is performed as follows: Freezing media is prepared by adding 1 ml of DMSO to 4 ml of complete DME/F-12 for a final product of 20% DMSO.

Cells are frozen as follows: a) Two 2 mL CellSeal vials are labeled to include processing date, passage number, donor identifier code, and cell count. Labeled cryovials are placed in a cryovial rack; b) Cells are pelleted by centrifugation at 500 g for 5 minutes at room temperature. Centrifugation is performed in 15 ml conical tubes; c) After the supernatant is removed, cells are re-suspended in 1 mL complete DME/F-12; d) Then, 1 ml of the 20% DMSO is added the cells at a rate of 10 drops per 30 seconds using an 18 gauge needle. This is based on the cell concentration to yield approximately 1 million cells per ml in a volume of approximately 2 ml of 10% DMSO; e) Using a syringe and 18 gauge needle, 1 mL of the cell mixture is drawn into the syringe. The sample is injected into the vial through puncturing the top septum of the vial. F) Using a heat sealer, seal both tubing segments. G) Place the vials into a box in box freezer and place in a −85 validated freezer. H) Vials are transferred to LN2 after 24 hours into a designated LN2 tank for cell vials only. In a sterile class II biologic safety within a class 10,000 clean production suite, cells from the two vials frozen at passage 0 are thawed under controlled conditions and each is washed in a 50 ml conical tube with 45 mL complete DME/F-12 (cDME/F-12) media (Hyclone) supplemented with 10% Fetal Bovine Serum from qualified dairy cattle herds with known negative pathogen pedigree (Atlas Biologicals) specified to have Endotoxin level: <=100 EU/ml (levels routinely <=10 EU/ml) and hemoglobin level: <=30 mg/dl (levels routinely <=25 mg/dl). The serum lot used is sequestered and one lot is used for all experiments. Cells are subsequently placed in two T-225 flasks containing 45 ml of cDME/F-12 and cultured for 24 hours at 37° C. at 5% CO2 in a fully humidified atmosphere. This allows the cells to adhere. Non-adherent cells are washed off using cDME/F-12 by gentle rinsing of the flask. The flask is then cultured for 4 days after which approximately 6.5M cells will be present per flask (passage 1). The cells from the flasks are split into ten T225s and cultured for 4 days (passage 2), after which it is split again to 50 T225 flasks (passage 3). This yields approximately 200 million cells. These cells are frozen down in vials containing ˜1M cells generating the master cell bank (on average 200 vials). After the testing panel from the master cell bank (MCB test panel) is received (see table 3), one vial from the master cell bank is thawed using the same protocol as the passage 0 cells, and placed into one T225 flask using cDME/F-12 with 10% FBS (passage 4). After culturing for 4-5 days, cells are split to five T225s and cultured for 4-5 days (passage 5). Cells are split again to 30 T225 and cultured for 4-5 days (passage 6). When cells are 70% confluent, they are frozen down in approximately 140 vials of 1 million cells per vial generating the working cell bank. When a patient dose is needed, one vial from the working cell bank is thawed and placed in one T225 flask (passage 7). After 4-5 days of culturing, cells are split to 5 T225 flasks (passage 8). After another 4-5 days of culturing, cells are split to 30 T225 flasks (passage 9). When the plates reach 70% confluence, cells are harvested for the clinical dose. The flasks yield approximately 120 million cells. Only 100 million cells are needed per clinical dose, and any extra cells will are used for release testing panel or are discarded. Cells are re-suspended in 10 mL of Isolyte S Multi-Electrolyte Solution. Then, ten milliliters of a 10% DMSO made with Isolyte S is added at a controlled rate over 5 minutes to the cells for a total of 20 mL of final product. The cell dose is packaged in a Charter CF-50 freezing bag, placed in a box in box freezing case and put in a validated −85° C. freezer. All processes in the generation, expansion, and product production are performed under conditions and testing that is compliant with current Good Manufacturing Processes and appropriate controls. Guidance issued by the FDA in 1998 Guidance for Industry: Guidance for Human Somatic Cell Therapy and Gene Therapy, the 2008 Guidance for FDA Reviewers and Sponsors Content and Review of Chemistry, Manufacturing, and Control (CMC) Information for Human Somatic Cell Therapy Investigational New Drug Applications (INDs), and the 1993 FDA points-to-consider document for master cell banks are all followed for the generation of the cell products described. The time elapsed from cell collection to storage is variable. A typical sample will take 2 weeks from time of collection until freezing for quarantine. Time cannot be calculated through final harvest because storage time is unknown. Storage time is based on need for the cells. Cells are stored as frozen cells according to the validated instructions for use for the Charter CF-50 bags. Some data concerning cell freezing and recovery are presented in the validation procedure for cell products. This is in agreement with other industry standards for storage of cell therapy products. Stability during cryopreservation is monitored using a comparison of pre-freeze and post-thaw data. The criterion tested is flow cytometry including viability and time to confluence. Prior to patient administration, doses will be sent to the administering facility frozen. The dose(s) will be sent in a dry shipper that will continuously monitor the temperature in route. Temperature data from shipment will be downloaded upon return of the dry shipper. Data will be shared with administering facility upon request. The facility will be responsible for the thawing of the cells. Once the cells are thawed a time limit of 6 hours has been established by which the cells must be administered. The temperature of 4 degrees Celsius must be maintained during storage of the thawed cells prior to administration. Charter CF-50 bags are filled with cellular product from cell bank that has been generated and tested according to the tests described in sections 1 and 2. Filling of Cryocyte bags will be performed by General Biotechnology with cells previously expanded from the working cell bank at passage 9. Cells are resuspended in 20 mL of Isolyte S Multi-Electrolyte Solution (B. Braun Medical) containing 10% DMSO. Each Charter CF-50 bag will contain 25, 50, or 100 million cells in a volume of 10 ml. Depending on indication, various doses may be used. For example, it may be possible to administer up to 400 million cells without observation of cell-mediated adverse events.

In order to test cell sterility, a variety of assays are known to one of skill in the art. Specifically, in one embodiment, a 2 mL aliquot of expended media from the culture is collected and placed into 2 SPS collection tubes (each tube containing 1 mL of the expended media). One tube is labeled for “Aerobic” and one tube is labeled for “Anaerobic” with a unique identifier for the sample. Samples are shipped to LABS, Inc for sterility testing. The USP/CFR 610.12 GMP (BASIC STERILITY) testing method is used for sterility. Bacteriostatic/Fungistatic activity uses the direct inoculation method. Cultures are incubated at LABS, Inc for two weeks for sterility screening. General BioTechnology received results within 3 weeks of shipment. For mycoplasma contamination testing, in process testing can completed using expended media from the MSC cultures can be tested for mycoplama using the Lonza Lucetta™ Luminometer with MycoAlert® Mycoplasma Detection Assay at General BioTechnology. MCB and final release testing is completed at Labs, Inc. Testing at Labs, Inc will test for the presence of agar cultivable and non-agar cultivable mycoplasma. The donors from which the cells are generated are extensively tested for infectious agents. The cells are cultured in a Class 10,000 clean room restricted to production of human cell products. At the MCB and WCB level, lytic and/or haemadsorbing viruses will be detected after inoculation using 3 sensitive indicator cell lines (specifically MRC-5, Vero and NBL-6 lines) with the MCB test article. This will be performed by a contract provider.

During manufacture of the cells of the invention, there are processes and procedures to ensure the quality of the product. These processes and procedures are validated and reviewed to continuously control the integrity of our products. The following are process control measures which maintain control over our product and are designed to prevent contamination or transmission of infectious disease. Standard Operating Procedures or written policies and procedures are developed and written with a standard format and are reviewed annually. Clinical outcomes are also monitored which collects patient data on adverse events of a patient. These events are part of the quality system internal assessment schedule to be reviewed as applicable events happen. Change control is procedures for how to properly implement changes. These changes are documented and approved. Materials used in the processing of our products are from qualified suppliers. Materials are received and handled according to our written procedures. Critical materials are traceable to the product as per our procedures. Equipment used for any purposes is maintained according to manufacturer guidelines and Good Laboratory Practices. Records are maintained of all maintenance and services rendered such as annual calibration. Equipment taken out of service is documented and return to service is also documented. Critical equipment is monitored according to our quality control and operational procedures. Cleaning and sanitation methods are defined for critical equipment. Equipment is validated for use before placed into service. Equipment is calibrated and maintained according to manufacturer's recommendations, regulatory requirements, and accrediting standards. Documentation is kept for each piece of equipment regarding identification number, repairs, scheduled calibration, and disposition. Critical equipment is traceable to the processing of an individual product. The manufacturing processes for the cells of the invention are qualified through validation of processes and procedures with the end goal of producing cell doses for use. Validation of the clean room was obtained through certification by Ace Lab Systems, Inc.

### Generation of Regenerative Cells

Cells are delivered frozen in DME/F-12 media with DMSO. Cells are thawed, washed by centrifugation according to protocol.

Cryocyte bags will be filled with cellular product from cell bank that has been generated and tested for mycoplasma contamination, sterility, viability and endotoxin content. Filling of Cryocyte bags will be performed by General Biotechnology with cells previously expanded from the working cell bank to passage 9. Cells are resuspended in 100 mL of Isolyte S Multi-Electrolyte Solution (B. Braun Medical). Each Cryocyte bag will contain 110 million cells in a volume of 100 ml. Approximately 110 million cells are needed per clinical dose, accounting for a 10% extra volume that may be lost due to spillage.

Manufacturing procedures take place in the General BioTechnology class 10,000 clean production suite. Each technician must properly gown when entering in the GMP room. Before entry into the clean lab area, the technician obtains a bunny suit in the ante room. After the hood of the bunny suit is placed on, they obtain a mouth covering and place on, making sure that all hair is fully covered under the hood and mouth covering. The technician then puts on a pair of sterile powder free gloves, and can enter the clean lab space with the thawed vial.

Environmental monitoring is performed in the Class 10,000 clean room. The clean room uses Biological Safety Cabinets (BSC) which maintains a Class 5 environment. BSC are certified annually by an outside qualified vender. Settling plates are performed quarterly with acceptable criteria based on USP. Two settling plates are placed in the BSC during processing for a minimum of 30 minutes. Also as a negative control, a covered settling plate will be placed inside the BSC at the same time After the settling plate has been in the BSC, evaluate the plate for presence of bacterial colonies, Colony Forming Units (cfu), by allowing the plate to incubate for 48 hours. Levels requiring alert are more than 1 colony per plate. Incubator temperature should be 36-38° C. TSA plates are used to evaluate the wide spectrum of possible bacteria present. Prepared plates stored in their original wrapping at 2-8° C. should be warmed to room temperature prior to use. The product is validated from the time of manufacture to be stable at room temperature (25° C.) for 192 h (8 days).

Additionally the clean room is monitored for room temperature and particle counts. Acceptable room temperature is between 15 and 30 degrees Celsius. A MetOne Aerocet 531 particle counter and is used to evaluate the particles in the air of the clean room. It is used to confirm that the number of loose particles in the air is less than 10,000 0.5 micron particles per ft³. The particle counter is run on a weekly basis in the three major areas of the clean room space. It runs for 30 minutes in the gowning area, on the counter inside the clean room space and inside the hood.

After each use of the clean room, the BSC is wiped down with 5.25% bleach then followed by a 70% isopropyl alcohol. Countertops inside the clean room space are wiped down with 70% isopropyl alcohol each day. Once a week all surfaces inside the clean room, including floor, are wiped down with enzymatic cleaner LpH using a dry disposable cloth. Yearly, all walls and ceiling are clean with a lint roller, and all soft walls are cleaned with 70% isopropyl alcohol.

Before laboratory technicians are allowed into the clean room, a gowning competency must be passed. RODAC plates are utilized to assess the competency of the technician. The acceptable limits of CFU/plate are listed in the table below. This is again repeated quarterly for all qualified technicians.

Cell isolation can begin with the delivery of the sample to the processing lab. Washing Tube containing the menstrual blood sample is topped up to 50 ml with PBS in the Biological Safety Cabinet and cells are washed by centrifugation at 500 g for 10 minutes at room temperature, which produced a cell pellet at the bottom of the conical tube. Under sterile conditions supernatant is decanted and the cell pellet is gently dissociated by tapping until the pellet appeared liquid. The pellet is resuspended in 50 ml of PBS and gently mixed so as to produce a uniform mixture of cells in PBS. The cells are washed again by centrifugation at 500 g for 10 minutes at room temperature. Under sterile conditions, the supernatant is decanted and the cell pellet is resuspended in 15 mL complete DME/F-12 media (Hyclone) supplemented with 10% Fetal Bovine Serum (Atlas Biologicals specified to have Endotoxin level: <=100 EU/ml (levels routinely <=10 EU/ml) and hemoglobin level: <=30 mg/dl (levels routinely <=25 mg/dl). The serum lot used is sequestered and one lot is used for all experiments. Additionally, the media is supplemented with 1% penicillin/streptomycin and 0.1% amphotericin B. The sample is then plated in a T75 flask and placed in a 37° C. incubator. Media is changed after 24 hours, and then every 2-3 days at the discretion of the laboratory staff.

Once cells reach 70-80% confluence they are passaged for expansion after which they are frozen down for quarantine (minimum 2 vials) and a culture screen will be completed. The expended media from the culture will be sent for sterility and mycoplasma testing. Cells from the sample are aliquotted and frozen in Cryocyte bags at a concentration of 110 million cells per bag.

Screening and collection occurs at a desired facility and is performed under approval of the local IRB. Donors are screened according to federal regulation 21 CFR1271 regarding allogeneic cell product. Specifically, healthy, non-smoking, female volunteers between 18-30 years of age sign informed consent form for providing endometrial tissue sample. The volunteers undergo a standard medical history and examination including evaluation for malignancy, diabetes, leukemia, heart disease. Hematology, biochemistry, and physical examination require no abnormalities. Patients are required to be negative for anti-HIV-1, HIV-2, hepatitis B surface antigen, hepatitis B core antibody, Treponema pallidum (syphilis), CJD, antibody to trypanosome cruzi, anti-HTLV-Il, Gonorrhea and Chlamydia. A collection of raw laboratory data will remain at the site and a donor case report forms are available for inspection.

Exclusion criteria are as follows: History of Toxic Shock Syndrome, Current tobacco use, Diabetes, Positive Communicable Disease Screen (Hepatitis B or C, syphilis, chlamydia, HIV, chlamydia, gonorrhea), Alcohol or drug abuse, and Unable to disclose health history of blood-related relatives.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of making an insulin-producing cell derived from an endometrial stromal stem cell (ESSC), the method comprising the steps of:
a. contacting at least one ESSC with a first cell culture medium comprising 20-30 mmol/l glucose, 5-15% FBS and 10⁻⁵-10⁻⁷ mol/l retinoic acid, and incubating the at least one cell for about 12-36 hours; then
b. contacting the at least one ESSC with a second cell culture medium comprising 20-30 mmol/l glucose and 5-15% FBS and incubating the at least one ESSC for about 1-4 days; then
c. contacting the at least one ESSC with ECM gel from Engelbreth-Holm-Swarm murine sarcoma and a third cell culture medium comprising 3-10 mmol/l glucose, 5-15% FBS, 3-30 mmol/l nicotinamide, 5-50 ng/ml epidermal growth factor, 5-500 ng/ml of FGF-10, and 50-600 nmol/l (−)-indolactam V and incubating the at least one ESSC for about 5-15 days; then
d. contacting the at least one ESSC with a fourth cell culture medium comprising 5-15% FBS, 1-100 nmol/l exendin-4, and 5-500 ng/ml Activin A and incubating the at least one ESSC for about 3-15 days; thereby deriving an insulin-producing cell from an ESSC.

2. The method of claim 1, wherein the ESSC is obtained from at least one biological sample selected from the group consisting of endometrium, endometrial stroma, endometrial membrane, and menstrual blood.

3. The method of claim 1, wherein the ESSC is a human ESSC.

4. The method of claim 1, wherein the first cell culture medium comprises 25 mmol/l glucose, 10% FBS and 10⁻⁶ mol/l retinoic acid, and the at least one ESSC is incubated in the first cell culture medium for about 24 hours.

5. The method of claim 1, wherein the second cell culture medium comprises 25 mmol/l glucose and 10% FBS and the at least one ESSC is incubated in the second cell culture medium for about 2 days.

6. The method of claim 1, wherein the third cell culture medium comprises 5.56 mmol/l glucose, 10% FBS, 10 mmol/l nicotinamide, 20 ng/ml epidermal growth factor, 50 ng/ml of FGF-10, and 300 nmol/l (−)-indolactam V and the at least one ESSC is incubated in the third cell culture medium for about 9 days.

7. The method of claim 1, wherein the fourth cell culture medium comprises 10% FBS, 10 nmol/l exendin-4, and 50 ng/ml Activin A and the at least one ESSC is incubated in the first cell culture medium for about 7 days.

8. A composition comprising an insulin-producing cell derived from an ESSC by the method of claim 1.

9. The composition of claim 8, wherein the ESSC is obtained from at least one biological sample selected from the group consisting of endometrium, endometrial stroma, endometrial membrane, and menstrual blood.

10. The composition of claim 8, wherein the ESSC is a human ESSC.

11. The composition of claim 8, wherein the insulin-producing cell exhibits at least one β cell marker selected from the group consisting of insulin, PAX4, PDX1, and GLUT2.

12. A method of treating a subject having diabetes comprising the steps of: administering at least one insulin-producing cell derived from an ESSC to the subject, wherein the insulin-producing cell secretes insulin within the subject, thereby treating the subject having diabetes.

13. The method of claim 12, wherein the ESSC is obtained from at least one biological sample selected from the group consisting of endometrium, endometrial stroma, endometrial membrane, and menstrual blood.

14. The method of claim 12, wherein the ESSC is a human ESSC.

15. The method of claim 12, wherein the ESSC is obtained from the subject.

16. The method of claim 12, wherein the diabetes is at least one selected from the group consisting of diabetes type I, diabetes type II and gestational diabetes.

17. The method of claim 12, wherein the at least one insulin-producing cell is administered by parenteral injection.

18. The method of claim 12, wherein the insulin-producing cell is derived from an ESSC according to the method of claim 1.

19. The method of claim 12, wherein the insulin-producing cell exhibits at least one β cell marker selected from the group consisting of insulin, PAX4, PDX1, and GLUT2.

20-27. (canceled)


SEQUENCE LISTING (14 sequences)

SEQ ID NO: 1 - DNA, 19 bp, Artificial sequence
  Chemically synthesized
gccctccaggacaggctgc

SEQ ID NO: 2 - DNA, 19 bp, Artificial sequence
  Chemically synthesized
ctccacctgccccacctgc

SEQ ID NO: 3 - DNA, 20 bp, Artificial sequence
  Chemically synthesized
gcatgtccgcgctccgctta

SEQ ID NO: 4 - DNA, 20 bp, Artificial sequence
  Chemically synthesized
tggagctgccctcgtcctgc

SEQ ID NO: 5 - DNA, 20 bp, Artificial sequence
  Chemically synthesized
accaccacctcccggctcag

SEQ ID NO: 6 - DNA, 20 bp, Artificial sequence
  Chemically synthesized
gcgcgtccgcttgttctcct

SEQ ID NO: 7 - DNA, 20 bp, Artificial sequence
  Chemically synthesized
gcgaccagaagcccgctgag

SEQ ID NO: 8 - DNA, 19 bp, Artificial sequence
  Chemically synthesized
gttcccccgtgtcgagtgg

SEQ ID NO: 9 - DNA, 20 bp, Artificial sequence
  Chemically synthesized
cagctggccatcgtcacggg

SEQ ID NO: 10 - DNA, 20 bp, Artificial sequence
  Chemically synthesized
ggctcgcacaccagacaggc

SEQ ID NO: 11 - DNA, 20 bp, Artificial sequence
  Chemically synthesized
acagagcctcgcctttgccg

SEQ ID NO: 12 - DNA, 20 bp, Artificial sequence
  Chemically synthesized
caccatcacgccctggtgcc

SEQ ID NO: 13 - DNA, 20 bp, Artificial sequence
  Chemically synthesized
ggggtacccacccagggacc

SEQ ID NO: 14 - DNA, 20 bp, Artificial sequence
  Chemically synthesized
cctgggaagcacctggcagc